(19) United States
(12) Patent Application Publication
(10) Pub. No.: US 2026/0298857 A1
(43) Pub. Date: Oct. 1, 2026

(54) GAS SENSOR

(71) Applicant: TDK Corporation, Tokyo (JP)

(72) Inventor: Makio KONDO, Tokyo (JP)

(21) Appl. No.: 19/569,537

(22) Filed: Mar. 17, 2026

(30) Foreign Application Priority Data
Mar. 25, 2025 (JP) 2025-049306

(51) Int. Cl. G01N 27/14; G01N 1/44; G01N 33/00

(52) U.S. Cl. CPC G01N 27/14; G01N 1/44; G01N 33/0027

(57) ABSTRACT

Disclosed herein is a gas sensor that includes a heater, a temperature-sensing element whose temperature varies in response to a change in a temperature of the heater, and a signal processing circuit that is configured to: calculate a concentration of a first gas based on an output signal derived at least from the temperature-sensing element obtained while the heater is maintained at a first temperature range when a level of the output signal satisfies a first condition while the heater is maintained at the first temperature range; and calculate a concentration of a second gas based on the output signal when the level of the output signal does not satisfy the first condition while the heater is maintained at the first temperature range. The first gas and the second gas have opposite thermal conductivities with respect to air in the first temperature range.

### CROSS-REFERENCE TO RELATED APPLICATION

This application claims the benefit of Japanese Patent Application No. 2025-049306, filed on Mar. 25, 2025, the entire disclosure of which is incorporated by reference herein.

### BACKGROUND OF THE ART

### Field of the Art

The present disclosure relates to a gas sensor, and more particularly, to a gas sensor capable of measuring concentrations of a plurality of types of target gases.

### Description of Related Art

JP 2017-009472A discloses a gas sensor including a thermistor and a heater heating the thermistor.

However, the gas sensor described in JP 2017-009472A is capable of measuring only one type of target gas.

### SUMMARY

A gas sensor according to an aspect of the present disclosure includes: a first heater; a first temperature-sensing element whose temperature varies in response to a change in a temperature of the first heater ; and a signal processing circuit. The signal processing circuit is configured to: calculate a concentration of a first gas based on an output signal derived at least from the first temperature-sensing element obtained while the first heater is maintained at a first temperature range when a level of the output signal satisfies a first condition while the first heater is maintained at the first temperature range; and calculate a concentration of a second gas based on the output signal when the level of the output signal does not satisfy the first condition while the first heater is maintained at the first temperature range. The first gas and the second gas have opposite thermal conductivities with respect to air in the first temperature range.

A gas sensor according to another aspect of the present disclosure includes: a first heater; a second heater; a first temperature-sensing element whose temperature varies in response to a change in a temperature of the first heater; a second temperature-sensing element whose temperature varies in response to a change in a temperature of the second heater; and a signal processing circuit. The signal processing circuit may be configured to: calculate a concentration of a first gas based on a first output signal derived from the first temperature-sensing element and the second temperature-sensing element while the first heater is maintained at a first temperature range and the second heater is maintained at a fifth temperature range different from the first temperature range when a level of a second output signal derived from the second temperature-sensing element satisfies a first condition while the second heater is maintained at the fifth temperature range; and calculate a concentration of a second gas based on the first output signal when a level of the second output signal does not satisfy the first condition while the second heater is maintained at the fifth temperature range. A thermal conductivity of the first gas in the fifth temperature range and a thermal conductivity of the second gas in the fifth temperature range are different from each other.

### BRIEF DESCRIPTION OF THE DRAWINGS

The above features and advantages of the present disclosure will be more apparent from the following description of some embodiments taken in conjunction with the accompanying drawings, in which:

FIG. 1 is a circuit diagram illustrating the configuration of a gas sensor 100 according to a first embodiment of the technology described herein;

FIG. 2 is a flowchart illustrating a first operation mode of the gas sensor 100;

FIGS. 3A to 3C are graphs illustrating changes in the concentrations of H₂ gas and CO₂ gas, a change in the gas detection signal Vgas, and a change in the output signal Vout, respectively, in the first operation mode of the gas sensor 100,;

FIG. 4 is a flowchart illustrating a second operation mode of the gas sensor 100;

FIGS. 5A to 5C are graphs illustrating changes in the concentrations of H₂ gas and CO₂ gas, a change in the gas detection signal Vgas, and a change in the output signal Vout, respectively, in the second operation mode of the gas sensor 100;

FIG. 6 is a flowchart illustrating a third operation mode of the gas sensor 100;

FIGS. 7A to 7C are graphs illustrating changes in the concentrations of H₂ gas and CO₂ gas, a change in the gas detection signal Vgas, and a change in the output signal Vout, respectively, in the third operation mode of the gas sensor 100;

FIGS. 8A to 8C are graphs illustrating changes in the concentrations of H₂ gas and CO₂ gas, a change in the gas detection signal Vgas, and a change in the output signal Vout, respectively, in the third operation mode of the gas sensor 100;

FIG. 9 is a flowchart illustrating a fourth operation mode of the gas sensor 100;

FIGS. 10A to 10C are graphs illustrating changes in the concentrations of H₂ gas and CO₂ gas, a change in the gas detection signal Vgas, and a change in the output signal Vout, respectively, in the fourth operation mode of the gas sensor 100;

FIGS. 11A to 11C are graphs illustrating changes in the concentrations of H₂ gas and CO₂ gas, a change in the gas detection signal Vgas, and a change in the output signal Vout, respectively, in the fourth operation mode of the gas sensor 100;

FIG. 12 is a circuit diagram illustrating the configuration of a gas sensor 200 according to a second embodiment of the technology described herein;

FIG. 13 is a flowchart illustrating a first operation mode of the gas sensor 200;

FIG. 14 is a flowchart illustrating a second operation mode of the gas sensor 200;

FIG. 15 is a flowchart illustrating a third operation mode of the gas sensor 200;

FIG. 16 is a flowchart illustrating a fourth operation mode of the gas sensor 200;

FIG. 17 is a circuit diagram illustrating the configuration of a gas sensor 200A according to a first modification of the second embodiment;

FIG. 18 is a circuit diagram illustrating the configuration of a gas sensor 200B according to a second modification of the second embodiment;

FIG. 19 is a circuit diagram illustrating a gas sensor 300 according to a third embodiment of the technology described herein;

FIG. 20 is a flowchart illustrating a first operation mode of the gas sensor 300;

FIGS. 21A to 21D are graphs illustrating changes in the concentrations of H₂ gas and CO₂ gas, a change in the gas detection signal Vgas, a change in the resistance value r1 of the thermistor Rd2, and a change in the output signal Vout, respectively, in the first operation mode of the gas sensor 300;

FIG. 22 is a flowchart illustrating a second operation mode of the gas sensor 300;

FIGS. 23A to 23D are graphs illustrating changes in the concentrations of H₂ gas and CO₂ gas, a change in the gas detection signal Vgas, a change in the resistance value r1 of the thermistor Rd2, and a change in the output signal Vout, respectively, in the second operation mode of the gas sensor 300;

FIG. 24 is a flowchart illustrating a third operation mode of the gas sensor 300;

FIGS. 25A to 25D are graphs illustrating changes in the concentrations of H₂ gas and CO₂ gas, a change in the gas detection signal Vgas, a change in the resistance value r1 of the thermistor Rd2, and a change in the output signal Vout, respectively, in the third operation mode of the gas sensor 300;

FIGS. 26A to 26D are graphs illustrating changes in the concentrations of H₂ gas and CO₂ gas, a change in the gas detection signal Vgas, a change in the resistance value r1 of the thermistor Rd2, and a change in the output signal Vout, respectively, in the third operation mode of the gas sensor 300;

FIG. 27 is a flowchart illustrating a fourth operation mode of the gas sensor 300;

FIGS. 28A to 28D are graphs illustrating changes in the concentrations of H₂gas and CO₂ gas, a change in the gas detection signal Vgas, a change in the resistance value r1 of the thermistor Rd2, and a change in the output signal Vout, respectively, in the third operation mode of the gas sensor 300;

FIGS. 29A to 29D are graphs illustrating changes in the concentrations of H₂ gas and CO₂ gas, a change in the gas detection signal Vgas, a change in the resistance value r1 of the thermistor Rd2, and a change in the output signal Vout, respectively, in the third operation mode of the gas sensor 300;

FIG. 30 is a circuit diagram illustrating the configuration of a gas sensor 300A according to a first modification of the third embodiment; and

FIG. 31 is a circuit diagram illustrating the configuration of a gas sensor 300B according to a second modification of the third embodiment.

### DETAILED DESCRIPTION OF THE EMBODIMENTS

The present disclosure describes a gas sensor capable of measuring a plurality of types of target gases.

Some embodiments of the present disclosure will be explained below in detail with reference to the accompanying drawings.

### First Embodiment

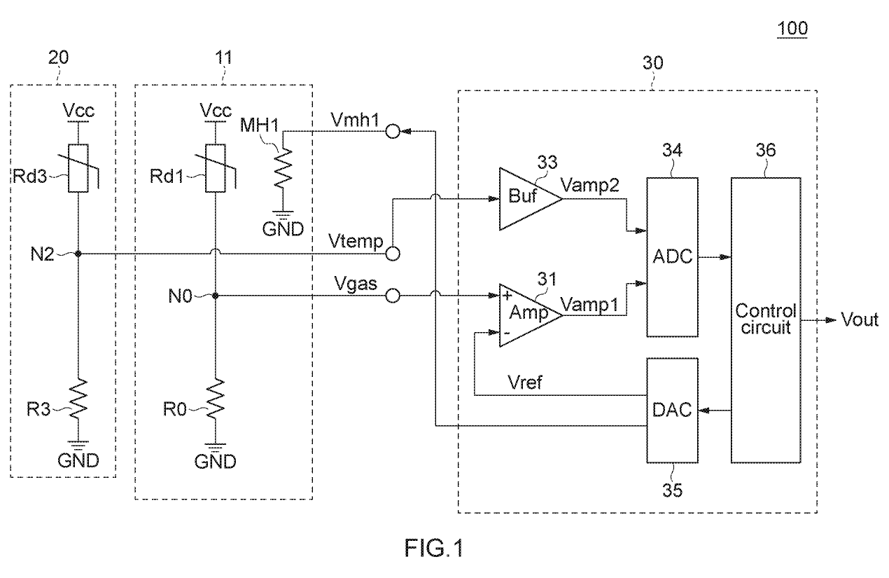

FIG. 1 is a circuit diagram illustrating the configuration of a gas sensor 100 according to a first embodiment of the technology described herein.

As illustrated in FIG. 1, the gas sensor 100 according to the first embodiment includes a sensor part 11 that generates a gas detection signal Vgas according to the concentration of a target gas, a temperature sensor 20 that generates a temperature detection signal Vtemp according to the ambient temperature, and a signal processing circuit 30. Although not particularly limited, the gas sensor 100 according to the first embodiment is a heat conduction type gas sensor for detecting the concentrations of hydrogen (H₂) gas and carbon dioxide (CO₂) gas in the measurement atmosphere.

The sensor part 11 includes a thermistor Rd1 and a fixed resistor R0, which are connected in series in this order between a power supply Vcc and a ground GND, and a heater MH1. The thermistor Rd1 is a temperature-sensing element whose temperature varies in response to a change in the temperature of the heater MH1. The gas detection signal Vgas output from the sensor part 11 appears at a node N0 between the thermistor Rd1 and the fixed resistor R0. The gas detection signal Vgas is an output signal derived at least from the thermistor Rd1. The thermistor Rd1 is a resistor whose resistance varies with temperature. Examples of the material of the thermistor Rd1 include vanadium oxide, amorphous silicon, polycrystalline silicon, an oxide with a spinel crystal structure containing manganese, titanium oxide, and yttrium-barium-copper oxide. The thermistor Rd1 may be an NTC thermistor having a negative temperature coefficient of resistance.

The temperature sensor 20 includes a thermistor Rd3 and a fixed resistor R3 which are connected in series between the power supply Vcc and the ground GND. The temperature detection signal Vtemp output from the temperature sensor 20 appears at a node N2 between the thermistor Rd3 and the fixed resistor R3. The temperature sensor 20 detects an environmental temperature. The environmental temperature is a temperature in the measurement atmosphere. The temperature sensor 20 may be designed so as not to be affected or so as to be hardly affected by heating by, for example, the heater MH1.

The signal processing circuit 30 includes a differential amplifier 31, a buffer 33, an AD converter (ADC) 34, a DA converter (DAC) 35, and a control circuit 36.

The differential amplifier 31 compares the gas detection signal Vgas and a reference potential Vref to generate an amplification signal Vamp1 corresponding to the amplified level difference (= Vgas – Vref) between the gas detection signal Vgas and the reference potential Vref. The buffer 33 buffers the temperature detection signal Vtemp to generate an amplification signal Vamp2. The amplification signals Vamp1 and Vamp2 are input to the AD converter 34. The AD converter 34 AD converts the amplification signals Vamp1 and Vamp2 to generate corresponding digital values and supplies them to the control circuit 36.

The control circuit 36 calculates the concentration of H₂ gas or CO₂ gas as a target gas based on the AD converted amplification signal Vamp1 and generates an output signal Vout indicating the concentration of H₂ gas or CO₂ gas. The control circuit 36 may calculate the H₂ or CO₂gas concentration using a calculation formula set therein. Further, the control circuit 36 supplies digital values of various control parameters to the DA converter 35. The DA converter 35 DA converts the digital values of the various control parameters to generate heater voltage Vmh1 and the reference potential Vref. The heater voltage Vmh1 is applied to the heater MH1, whereby the heater MH1 is heated. The reference potential Vref is supplied to the differential amplifier 31.

The heater MH1 is heated to a first temperature range by application of a heater voltage Vmh1 when the H₂ gas concentration is measured, and to a second temperature range when the CO₂ gas concentration is measured. The first temperature range is a range in which the difference between the thermal conductivity of air and that of H₂ gas is large. The second temperature range is a range in which the difference between the thermal conductivity of air and that of CO₂ gas is large. The term “temperature range” in the present specification refers to a range having, for example, a width of 1° C. or less. The first and second temperature ranges may be different from each other. The first temperature range is a predetermined temperature range included within a range of 80° C. or higher and 120° C. or lower, for example, a temperature range around 100°C.. The second temperature range is a predetermined temperature range included within a range of 130° C. or higher and 230° C. or lower, for example, a temperature range around 150° C..

When H₂ or CO₂ gas is present in the measurement atmosphere while the heater MH1 is being heated, the heat dissipation characteristics of the heater MH1 change according to the concentration of H₂ or CO₂ gas. This change appears as a change in the temperature of the thermistor Rd1, i.e., a change in the resistance of the thermistor Rd1. In the first and second temperature ranges, the thermal conductivity of H₂ gas is higher than that of air, whereas the thermal conductivity of CO₂ gas is lower than that of air. That is, H₂ gas and CO₂ gas exhibit opposite relative thermal conductivities with respect to air in the first and second temperature ranges. In the first temperature range, since the difference between the thermal conductivity of air and that of H₂ gas is large, a change in the temperature of the thermistor Rd1 per change in the concentration of H₂ gas, i.e., a change in the resistance of the thermistor Rd1 increases. That is, the gas sensor 100 can detect the concentration of H₂ gas with high sensitivity while the heater MH1 is maintained at the first temperature range. In the second temperature range, since the difference between the thermal conductivity of air and that of CO₂ gas is large, a change in the temperature of the thermistor Rd1 per change in the concentration of CO₂ gas, i.e., a change in the resistance of the thermistor Rd1 increases. That is, the gas sensor 100 can detect the concentration of CO₂ gas with high sensitivity while the heater MH1 is maintained at the second temperature range.

Therefore, when H₂ gas is present in the measurement atmosphere while the heater MH1 is maintained at the first or second temperature range by application of the heater voltage Vmh1, the heat dissipation characteristics of the heater MH1 increase as the H₂ gas concentration increases, so that the temperature of the heater MH1 decreases and, consequently, the temperature of the thermistor Rd1 also decreases. Thus, the resistance of the thermistor Rd1 increases as the H₂ gas concentration increases, so that the level of the gas detection signal Vgas decreases.

On the other hand, when CO₂ gas is present in the measurement atmosphere while the heater MH1 is maintained at the first or second temperature range by application of the heater voltage Vmh1, the heat dissipation characteristics of the heater MH1 decrease as the CO₂ gas concentration increases, so that the temperature of the heater MH1 increases and, consequently, the temperature of the thermistor Rd1 also increases. Thus, the resistance of the thermistor Rd1 decreases as the CO₂ gas concentration in the measurement atmosphere increases, so that the level of the gas detection signal Vgas increases.

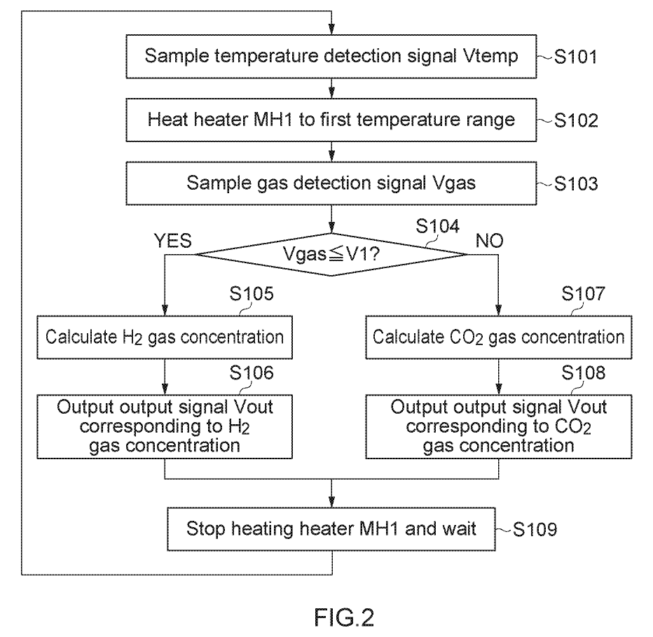

FIG. 2 is a flowchart illustrating a first operation mode of the gas sensor 100. The first operation mode of the gas sensor 100 is a mode based on measuring the H₂ gas concentration in the measurement atmosphere by heating the heater MH1 to the first temperature range. In this mode, when the CO₂ gas concentration in the measurement atmosphere increases in a state where the H₂gas concentration in the measurement atmosphere is sufficiently low, the target gas is automatically switched from H₂ gas to CO₂ gas.

First, in the first operation mode of the gas sensor 100, the signal processing circuit 30 samples the temperature detection signal Vtemp to calculate the ambient temperature (step S101). Subsequently, the control circuit 36 included in the signal processing circuit 30 outputs a heater instruction value calculated based on the ambient temperature to the DA converter 35 to heat the heater MH1 to the first temperature range (step S102). The heater instruction value is converted into the heater voltage Vmh1 by the DA converter 35, which is then applied to the heater MH1. Then, the signal processing circuit 30 samples the gas detection signal Vgas while the heater MH1 is maintained at the first temperature range (step S103).

Then, the signal processing circuit 30 determines whether the level of the gas detection signal Vgas is equal to or lower than a threshold value V1 (step S104). This determination corresponds to determining whether the target gas is switched from H₂ gas to CO₂ gas. That is, in the mode for measuring the H₂ gas concentration in the measurement atmosphere, the level of the gas detection signal Vgas decreases as the H₂ gas concentration increases, so that the actual level of the gas detection signal Vgas should be equal to or lower than the level of the gas detection signal Vgas that is to be obtained when the H₂ gas concentration is around zero. The threshold value V1 corresponds to the level of the gas detection signal Vgas to be obtained when the H₂ gas concentration is around zero. Therefore, when the condition that the level of the gas detection signal Vgas is equal to or lower than the threshold value V1 is satisfied (Vgas ≤ V1), the H₂ gas concentration is calculated based on the gas detection signal Vgas (step S105), and the output signal Vout corresponding to the H₂ gas concentration is output to the outside (step S106). The H₂ gas concentration may be calculated using a calculation formula for H₂ gas set in the control circuit 36. Thereafter, the heating of the heater MH1 is stopped, and waiting is performed until the next measurement timing (step S109). When the next measurement timing arrives, the flow returns to step S101 for repetition of the above operation.

On the other hand, when the condition that the level of the gas detection signal Vgas is equal to or lower than the threshold value V1 is not satisfied (Vgas > V1), it is suggested that CO₂gas is present at a high concentration in the measurement atmosphere. In this case, the CO₂ gas concentration is calculated based on the gas detection signal Vgas (step S107), and the output signal Vout corresponding to the CO₂ gas concentration is output to the outside (step S108). The CO₂ gas concentration may be calculated using a calculation formula for CO₂ gas set in the control circuit 36. Thereafter, the heating of the heater MH1 is stopped, and waiting is performed until the next measurement timing (step S109). When the next measurement timing arrives, the flow returns to step S101 for repetition of the above operation.

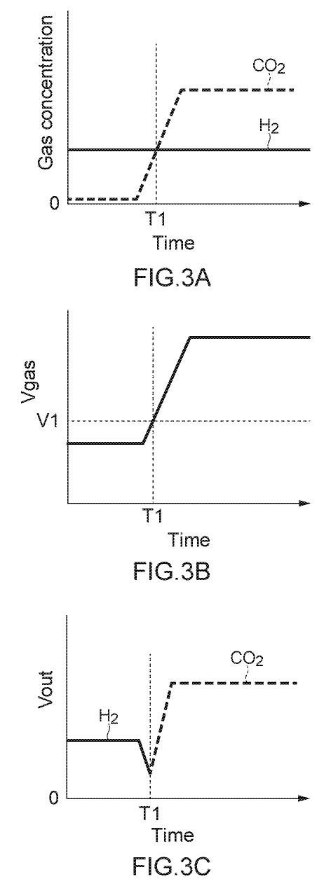

FIGS. 3A to 3C are graphs illustrating changes in the concentrations of H₂ gas and CO₂ gas, a change in the gas detection signal Vgas, and a change in the output signal Vout, respectively, in the first operation mode of the gas sensor 100.

In the example illustrated in FIG. 3A, the H₂ gas concentration is constant, whereas the CO₂ gas concentration sharply increases around time T1. In this case, as illustrated in FIG. 3B, the level of the gas detection signal Vgas is equal to or lower than the threshold value V1 until time T1 and is higher than the threshold value V1 after time T1. As a result, as illustrated in FIG. 3C, the signal processing circuit 30 generates the output signal Vout indicating the H₂gas concentration until time T1 and generates the output signal Vout indicating the CO₂ gas concentration after time T1.

As described above, in the first operation mode of the gas sensor 100, the H₂ gas concentration in the measurement atmosphere is primarily measured, and when the CO₂ gas concentration in the measurement atmosphere increases, the target gas is automatically switched from H₂ gas to CO₂ gas. Then, when the CO₂ gas concentration in the measurement atmosphere decreases, the target gas is switched from CO₂ gas to H₂ gas. In the first operation mode of the gas sensor 100, the heater MH1 is heated to the first temperature range optimized for the measurement of the H₂ gas concentration, allowing the H₂ gas concentration in the measurement atmosphere to be measured with high sensitivity.

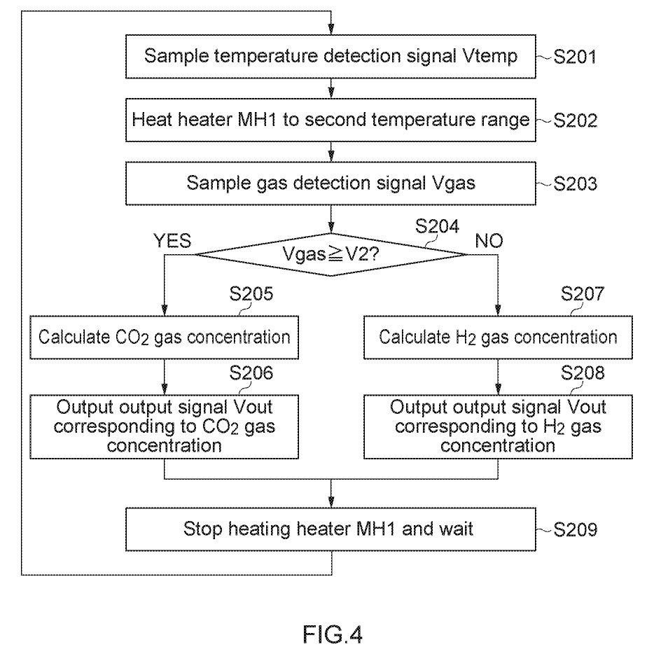

FIG. 4 is a flowchart illustrating a second operation mode of the gas sensor 100. The second operation mode of the gas sensor 100 is a mode based on measuring the CO₂ gas concentration in the measurement atmosphere by heating the heater MH1 to the second temperature range. In this mode, when the H₂ gas concentration in the measurement atmosphere increases in a state where the CO₂ gas concentration in the measurement atmosphere is sufficiently low, the target gas is automatically switched from CO₂ gas to H₂ gas.

First, in the second operation mode of the gas sensor 100, the signal processing circuit 30 samples the temperature detection signal Vtemp to calculate the ambient temperature (step S201). Subsequently, the control circuit 36 included in the signal processing circuit 30 outputs a heater instruction value calculated based on the ambient temperature to the DA converter 35 to heat the heater MH1 to the second temperature range (step S202). The heater instruction value is converted into the heater voltage Vmh1 by the DA converter 35, which is then applied to the heater MH1. Then, the signal processing circuit 30 samples the gas detection signal Vgas while the heater MH1 is maintained at the second temperature range (step S203).

Then, the signal processing circuit 30 determines whether the level of the gas detection signal Vgas is equal to or higher than a threshold value V2 (step S204). This determination corresponds to determining whether the target gas is switched from CO₂ gas to H₂ gas. That is, in the mode for measuring the CO₂gas concentration in the measurement atmosphere, the level of the gas detection signal Vgas increases as the CO₂ gas concentration increases, so that the actual level of the gas detection signal Vgas should be equal to or higher than the level of the gas detection signal Vgasthat is to be obtained when the CO₂ gas concentration is around the CO₂ gas concentration (e.g., 400 ppm) in the atmosphere under normal conditions. The threshold valve V2 corresponds to the gas detection signal Vgas that is to be obtained when the CO₂ gas concentration is around the CO₂ gas concentration (e.g., 400 ppm) in the atmosphere under normal conditions. Therefore, when the condition that the level of the gas detection signal Vgas is equal to or higher than the threshold value V2 is satisfied (Vgas ≥ V2), the CO₂ gas concentration is calculated based on the gas detection signal Vgas (step S205), and the output signal Vout corresponding to the CO₂ gas concentration is output to the outside (step S206). The CO₂ gas concentration may be calculated using the calculation formula for CO₂ gas set in the control circuit 36. Thereafter, the heating of the heater MH1 is stopped, and waiting is performed until the next measurement timing (step S209). When the next measurement timing arrives, the flow returns to step S201 for repetition of the above operation.

On the other hand, when the condition that the level of the gas detection signal Vgas is equal to or higher than the threshold value V2 is not satisfied (Vgas < V2), it is suggested that H₂ gas is present at a high concentration in the measurement atmosphere. In this case, the H₂gas concentration is calculated based on the gas detection signal Vgas (step S207), and the output signal Vout corresponding to the H₂ gas concentration is output to the outside (step S208). The H₂gas concentration may be calculated using the calculation formula for H₂ gas set in the control circuit 36. Thereafter, the heating of the heater MH1 is stopped, and waiting is performed until the next measurement timing (step S209). When the next measurement timing arrives, the flow returns to step S201 for repetition of the above operation.

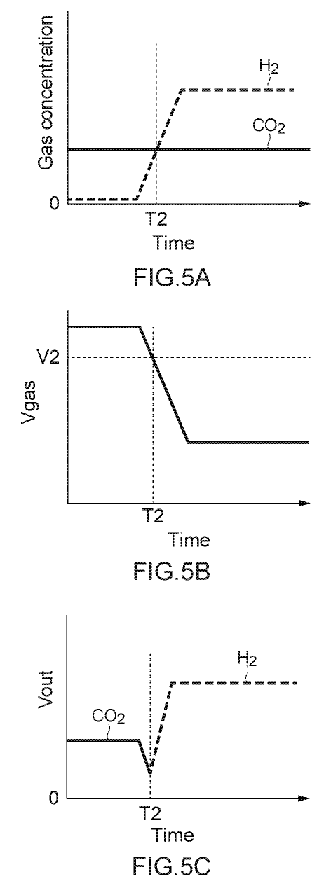

FIGS. 5A to 5C are graphs illustrating changes in the concentrations of H₂ gas and CO₂ gas, a change in the gas detection signal Vgas, and a change in the output signal Vout, respectively, in the second operation mode of the gas sensor 100.

In the example illustrated in FIG. 5A, the CO₂ gas concentration is constant, whereas the H₂ gas concentration sharply increases around time T2. In this case, as illustrated in FIG. 5B, the level of the gas detection signal Vgas is equal to or higher than the threshold value V2 until time T2 and is lower than the threshold value V2 after time T2. As a result, as illustrated in FIG. 5C, the signal processing circuit 30 generates the output signal Vout indicating the CO₂ gas concentration until time T2 and generates the output signal Vout indicating the H₂ gas concentration after time T2.

As described above, in the second operation mode of the gas sensor 100, the CO₂ gas concentration in the measurement atmosphere is primarily measured, and when the H₂ gas concentration in the measurement atmosphere increases, the target gas is automatically switched from CO₂gas to H₂ gas. Then, when the H₂ gas concentration in the measurement atmosphere decreases, the target gas is switched from H₂ gas to CO₂ gas. In the second operation mode of the gas sensor 100, the heater MH1 is heated to the second temperature range optimized for the measurement of the CO₂gas concentration, allowing the CO₂ gas concentration in the measurement atmosphere to be measured with high sensitivity.

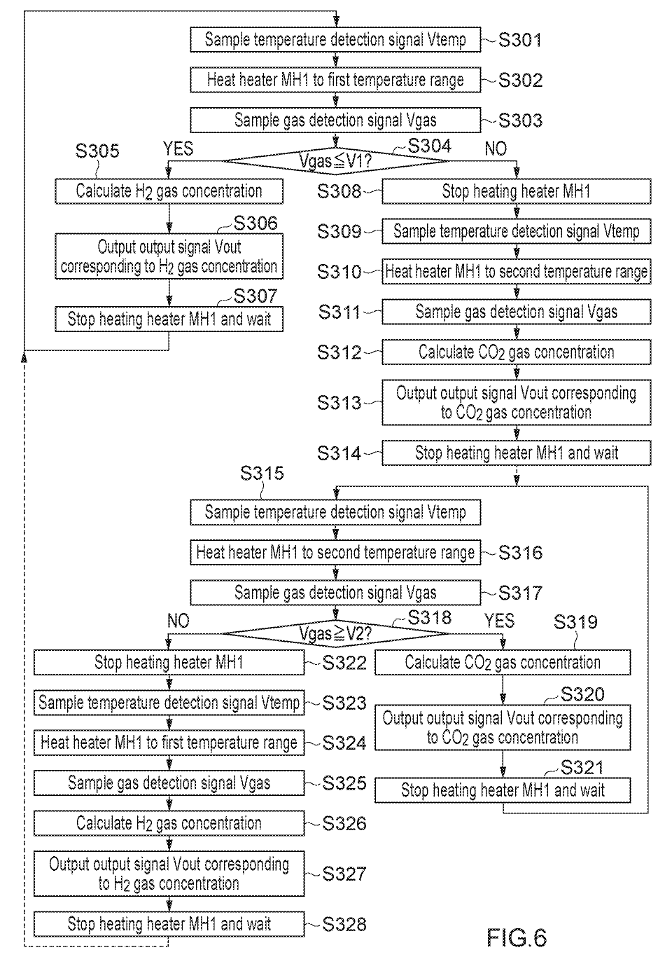

FIG. 6 is a flowchart illustrating a third operation mode of the gas sensor 100. The third operation mode of the gas sensor 100 is a mode in which the heater MH1 is heated to the first temperature range when the H₂ gas concentration in the measurement atmosphere is measured, and to the second temperature range when the CO₂ gas concentration is measured.

First, in the third operation mode of the gas sensor 100, when the H₂ gas concentration in the measurement atmosphere is measured, the signal processing circuit 30 samples the temperature detection signal Vtemp to calculate the ambient temperature (step S301). Subsequently, the control circuit 36 included in the signal processing circuit 30 outputs a heater instruction value calculated based on the ambient temperature to the DA converter 35 to heat the heater MH1 to the first temperature range (step S302). The heater instruction value is converted into the heater voltage Vmh1 by the DA converter 35, which is then applied to the heater MH1. Then, the signal processing circuit 30 samples the gas detection signal Vgas while the heater MH1 is maintained at the first temperature range (step S303).

Then, the signal processing circuit 30 determines whether the level of the gas detection signal Vgas is equal to or lower than the threshold value V1 (step S304). This determination corresponds to determining whether the target gas is switched from H₂ gas to CO₂ gas. Then, when the condition that the level of the gas detection signal Vgas is equal to or lower than the threshold value V1 is satisfied (Vgas ≤ V1), the H₂ gas concentration is calculated based on the gas detection signal Vgas (step S305), and the output signal Vout corresponding to the H₂ gas concentration is output to the outside (step S306). The H₂ gas concentration may be calculated using the calculation formula for H₂ gas set in the control circuit 36. Thereafter, the heating of the heater MH1 is stopped, and waiting is performed until the next measurement timing (step S307). When the next measurement timing arrives, the flow returns to step S301 for repetition of the above operation.

On the other hand, when the condition that the level of the gas detection signal Vgas is equal to or lower than the threshold value V1 is not satisfied (Vgas > V1), it is suggested that CO₂ gas is present at a high concentration in the measurement atmosphere. In this case, the heating of the heater MH1 is temporarily stopped (step S308), and the signal processing circuit 30 samples the temperature detection signal Vtemp again to calculate the ambient temperature (step S309). Subsequently, the control circuit 36 included in the signal processing circuit 30 outputs a heater instruction value calculated based on the ambient temperature to the DA converter 35 to heat the heater MH1 to the second temperature range (step S310). The heater instruction value is converted into the heater voltage Vmh1 by the DA converter 35, which is then applied to the heater MH1. Then, the signal processing circuit 30 samples the gas detection signal Vgas while the heater MH1 is maintained at the second temperature range (step S311).

Then, the CO₂ gas concentration is calculated based on the gas detection signal Vgas (step S312), and the output signal Vout corresponding to the CO₂ gas concentration is output to the outside (step S313). The CO₂ gas concentration may be calculated using the calculation formula for CO₂ gas set in the control circuit 36. Thereafter, the heating of the heater MH1 is stopped, and waiting is performed until the next measurement timing (step S314).

When the next measurement timing arrives, the signal processing circuit 30 samples the temperature detection signal Vtemp again to calculate the ambient temperature (step S315). Subsequently, the control circuit 36 included in the signal processing circuit 30 outputs a heater instruction value calculated based on the ambient temperature to the DA converter 35 to heat the heater MH1 to the second temperature range (step S316). The heater instruction value is converted into the heater voltage Vmh1 by the DA converter 35, which is then applied to the heater MH1. Then, the signal processing circuit 30 samples the gas detection signal Vgas while the heater MH1 is maintained at the second temperature range (step S317).

Then, the signal processing circuit 30 determines whether the level of the gas detection signal Vgas is equal to or higher than the threshold value V2 (step S318). This determination corresponds to determining whether the target gas is switched from CO₂ gas to H₂ gas. Then, when the condition that the level of the gas detection signal Vgas is equal to or higher than the threshold value V2 is satisfied (Vgas ≥ V2), the CO₂ gas concentration is calculated based on the gas detection signal Vgas (step S319), and the output signal Vout corresponding to the CO₂ gas concentration is output to the outside (step S320). The CO₂ gas concentration may be calculated using the calculation formula for CO₂ gas set in the control circuit 36. Thereafter, the heating of the heater MH1 is stopped, and waiting is performed until the next measurement timing (step S321). When the next measurement timing arrives, the flow returns to step S315 for repetition of the above operation.

On the other hand, when the condition that the level of the gas detection signal Vgas is equal to or higher than the threshold value V2 is not satisfied (Vgas < V2), it is suggested that H₂ gas is present at a high concentration in the measurement atmosphere. In this case, the heating of the heater MH1 is temporarily stopped (step S322), and the signal processing circuit 30 samples the temperature detection signal Vtemp again to calculate the ambient temperature (step S323). Subsequently, the control circuit 36 included in the signal processing circuit 30 outputs a heater instruction value calculated based on the ambient temperature to the DA converter 35 to heat the heater MH1 to the first temperature range (step S324). The heater instruction value is converted into the heater voltage Vmh1 by the DA converter 35, which is then applied to the heater MH1. Then, the signal processing circuit 30 samples the gas detection signal Vgas while the heater MH1 is maintained at the first temperature range (step S325).

Then, the H₂gas concentration is calculated based on the gas detection signal Vgas (step S326), and the output signal Vout corresponding to the H₂ gas concentration is output to the outside (step S327). The H₂ gas concentration may be calculated using the calculation formula for H₂ gas set in the control circuit 36. Thereafter, the heating of the heater MH1 is stopped, and waiting is performed until the next measurement timing (step S328). When the next measurement timing arrives, the flow returns to step S301 for repetition of the above operation.

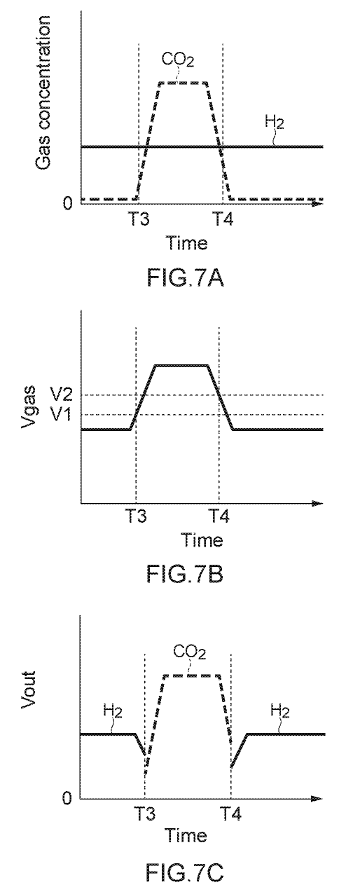

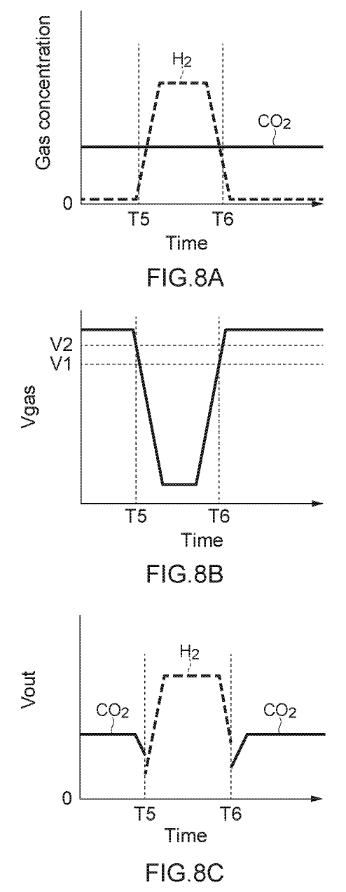

FIGS. 7A to 7C are graphs illustrating changes in the concentrations of H₂ gas and CO₂ gas, a change in the gas detection signal Vgas, and a change in the output signal Vout, respectively, in the third operation mode of the gas sensor 100. FIGS. 8A to 8C are graphs illustrating changes in the concentrations of H₂ gas and CO₂ gas, a change in the gas detection signal Vgas, and a change in the output signal Vout, respectively, in the third operation mode of the gas sensor 100.

In the example illustrated in FIG. 7A, the H₂ gas concentration is constant, whereas the CO₂ gas concentration sharply increases around time T3 and sharply decreases around time T4. In this case, as illustrated in FIG. 7B, the level of the gas detection signal Vgas is equal to or lower than the threshold value V1 until time T3; is higher than the threshold value V1 at time T3; and is equal to or lower than the threshold value V2 after time T4. As a result, as illustrated in FIG. 7C, until time T3, the signal processing circuit 30 repeats steps S301 to S307 to generate the output signal Vout indicating the H₂ gas concentration; from time T3 to time T4, the signal processing circuit 30 repeats steps S315 to S321 after executing steps S308 to S314 to generate the output signal Vout indicating the CO₂ gas concentration; and after time T4, the signal processing circuit 30 repeats steps S301 to S307 after executing steps S322 to S328 to generate again the output signal Vout indicating the H₂ gas concentration.

In the example illustrated in FIG. 8A, the CO₂ gas concentration is constant, whereas the H₂ gas concentration sharply increases around time T5 and sharply decreases around time T6. In this case, as illustrated in FIG. 8B, the level of the gas detection signal Vgas is equal to or higher than the threshold value V2 until time T5; is lower than the threshold value V2 at time T5; and is equal to or higher than the threshold value V1 after time T6. As a result, as illustrated in FIG. 8C, until time T5, the signal processing circuit 30 repeats steps S315 to S321 after executing steps S308 to S314 to generate the output signal Vout indicating the CO₂ gas concentration; from time T5 to time T6, the signal processing circuit 30 repeats steps S301 to S307 after executing steps S322 to S328 to generate the output signal Vout indicating the H₂ gas concentration; and after time T6, the signal processing circuit 30 repeats steps S315 to S321 after executing steps S308 to S314 to generate again the output signal Vout indicating the CO₂ gas concentration.

As described above, in the third operation mode of the gas sensor 100, when the CO₂ gas concentration in the measurement atmosphere increases during the measurement of the H₂ gas concentration, the target gas is automatically switched from H₂ gas to CO₂ gas; when the H₂ gas concentration in the measurement atmosphere increases during the measurement of the CO₂ gas concentration, the target gas is automatically switched from CO₂ gas to H₂ gas. In the third operation mode of the gas sensor 100, the heater MH1 is heated to the first temperature range optimized for the measurement of the H₂ gas concentration during the measurement of the H₂ gas concentration, while the heater MH1 is heated to the second temperature range optimized for the measurement of the CO₂ gas concentration during the measurement of the CO₂ gas concentration, allowing the concentrations of both H₂ gas and CO₂ gas in the measurement atmosphere to be measured with high sensitivity. Although, in the above example, the flow proceeds to step S315 when the next measurement timing arrives in step S314, the flow may proceed to step S301 in this case.

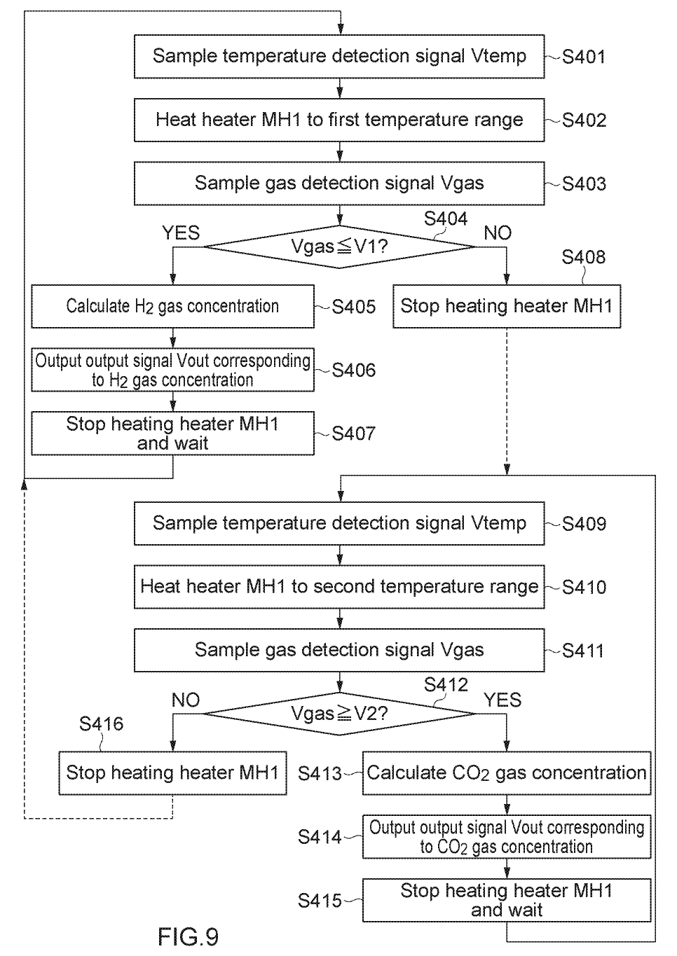

FIG. 9 is a flowchart illustrating a fourth operation mode of the gas sensor 100. In the fourth operation mode of the gas sensor 100, when the H₂ gas concentration in the measurement atmosphere is measured, the heater MH1 is heated to the first temperature range; whereas when the CO₂ gas concentration in the measurement atmosphere is measured, the heater MH1 is heated to the second temperature range, and a determination is made using the threshold value after the switching of the target gas and before the actual measurement of the concentration of the target gas.

First, in the fourth operation mode of the gas sensor 100, when the H₂ gas concentration in the measurement atmosphere is measured, the signal processing circuit 30 samples the temperature detection signal Vtemp to calculate the ambient temperature (step S401). Subsequently, the control circuit 36 included in the signal processing circuit 30 outputs a heater instruction value calculated based on the ambient temperature to the DA converter 35 to heat the heater MH1 to the first temperature range (step S402). The heater instruction value is converted into the heater voltage Vmh1 by the DA converter 35, which is then applied to the heater MH1. Then, the signal processing circuit 30 samples the gas detection signal Vgas while the heater MH1 is maintained at the first temperature range (step S403).

Then, the signal processing circuit 30 determines whether the level of the gas detection signal Vgas is equal to or lower than the threshold value V1 (step S404). This determination corresponds to determining whether the H₂ gas concentration in the measurement atmosphere is actually measured. Then, when the condition that the level of the gas detection signal Vgas is equal to or lower than the threshold value V1 is satisfied (Vgas ≤ V1), the H₂gas concentration is calculated based on the gas detection signal Vgas (step S405), and the output signal Vout corresponding to the H₂ gas concentration is output to the outside (step S406). The H₂gas concentration may be calculated using the calculation formula for H₂gas set in the control circuit 36. Thereafter, the heating of the heater MH1 is stopped, and waiting is performed until the next measurement timing (step S407). When the next measurement timing arrives, the flow returns to step S401 for repetition of the above operation.

On the other hand, when the condition that the level of the gas detection signal Vgas is equal to or lower than the threshold value V1 is not satisfied (Vgas > V1), it is suggested that CO₂ gas is present at a high concentration in the measurement atmosphere. In this case, the heating of the heater MH1 is temporarily stopped (step S408), and the target gas is switched from H₂gas to CO₂gas.

When the target gas is switched from H₂ gas to CO₂ gas, the signal processing circuit 30 samples the temperature detection signal Vtemp to calculate the ambient temperature (step S409). Subsequently, the control circuit 36 included in the signal processing circuit 30 outputs a heater instruction value calculated based on the ambient temperature to the DA converter 35 to heat the heater MH1 to the second temperature range (step S410). The heater instruction value is converted into the heater voltage Vmh1 by the DA converter 35, which is then applied to the heater MH1. Then, the signal processing circuit 30 samples the gas detection signal Vgas while the heater MH1 is maintained at the second temperature range (step S411).

Then, the signal processing circuit 30 determines whether the level of the gas detection signal Vgas is equal to or higher than the threshold value V2 (step S412). This determination corresponds to determining whether the CO₂ gas concentration in the measurement atmosphere is actually measured. Then, when the condition that the level of the gas detection signal Vgas is equal to or higher than the threshold value V2 is satisfied (Vgas ≥ V2), the CO₂ gas concentration is calculated based on the gas detection signal Vgas (step S413), and the output signal Vout corresponding to the CO₂ gas concentration is output to the outside (step S414). The CO₂ gas concentration may be calculated using the calculation formula for CO₂ gas set in the control circuit 36. Thereafter, the heating of the heater MH1 is stopped, and waiting is performed until the next measurement timing (step S415). When the next measurement timing arrives, the flow returns to step S409 for repetition of the above operation.

On the other hand, when the condition that the level of the gas detection signal Vgas is equal to or higher than the threshold value V2 is not satisfied (Vgas < V2), it is suggested that H₂ gas is present at a high concentration in the measurement atmosphere. In this case, the heating of the heater MH1 is temporarily stopped (step S416), and the flow returns to step S401, where the target gas is switched from CO₂ gas to H₂ gas.

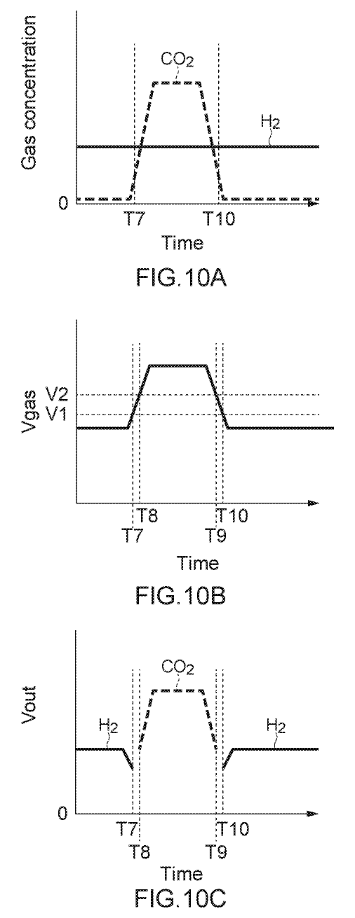

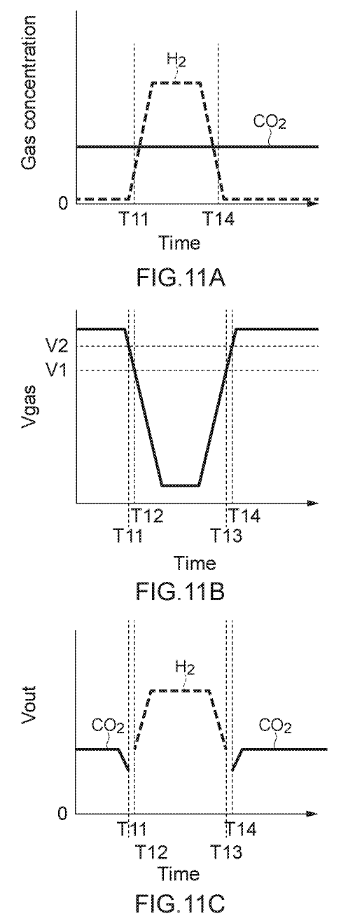

FIGS. 10A to 10C are graphs illustrating changes in the concentrations of H₂ gas and CO₂ gas, a change in the gas detection signal Vgas, and a change in the output signal Vout, respectively, in the fourth operation mode of the gas sensor 100. FIGS. 11A to 11C are graphs illustrating changes in the concentrations of H₂ gas and CO₂ gas, a change in the gas detection signal Vgas, and a change in the output signal Vout, respectively, in the fourth operation mode of the gas sensor 100.

In the example illustrated in FIG. 10A, the H₂ gas concentration is constant, whereas the CO₂gas concentration sharply increases around time T7 and sharply decreases around time T10. In this case, as illustrated in FIG. 10B, the level of the gas detection signal Vgas is equal to or lower than the threshold value V1 until time T7; is higher than the threshold value V1 but is lower than the threshold value V2 from time T7 to time T8; is equal to or higher than the threshold value V2 from time T8 to time T9; is higher than the threshold value V1 but is lower than the threshold value V2 from time T9 to time T10; and is equal to or lower than the threshold value V1 after time T10. As a result, as illustrated in FIG. 10C, until time T7, the signal processing circuit 30 repeats steps S401 to S407 to generate the output signal Vout indicating the H₂ gas concentration; from time T8 to time T9, the signal processing circuit 30 repeats steps S409 to S415 to generate the output signal Vout indicating the CO₂ gas concentration; and after time T10, the signal processing circuit 30 repeats steps S401 to S407 to generate again the output signal Vout indicating the H₂ gas concentration.

From time T7 to time T8, steps S401 to S404, steps S408 to S412, and step S416 are repeated, during which no output signal Vout is obtained. Similarly, from time T9 to time T10, steps S401 to S404, steps S408 to S412, and step S416 are repeated, during which no output signal Vout is obtained.

In the example illustrated in FIG. 11A, the CO₂ gas concentration is constant, whereas the H₂ gas concentration sharply increases around time T11 and sharply decreases around time T14. In this case, as illustrated in FIG. 11B, the level of the gas detection signal Vgas is equal to or higher than the threshold value V2 until time T11; is higher than the threshold value V1 but is lower than the threshold value V2 from time T11 to time T12; is equal to or lower than the threshold value V1 from time T12 to time T13; is higher than the threshold value V1 but is lower than the threshold value V2 from time T13 to time T14; and is equal to or higher than the threshold value V2 after time T14. As a result, as illustrated in FIG. 11C, until time T11, the signal processing circuit 30 repeats steps S409 to S415 to generate the output signal Vout indicating the CO₂ gas concentration; from time T12 to time T13, the signal processing circuit 30 repeats steps S401 to S407 to generate the output signal Vout indicating the H₂ gas concentration; and after time T14, the signal processing circuit 30 repeats steps S409 to S415 to generate again the output signal Vout indicating the CO₂ gas concentration.

From time T11 to time T12, steps S401 to S404, steps S408 to S412, and step S416 are repeated, during which no output signal Vout is obtained. Similarly, from time T13 to time T14, steps S401 to S404, steps S408 to S412, and step S416 are repeated, during which no output signal Vout is obtained.

As described above, similarly to the third operation mode of the gas sensor 100, in the fourth operation mode, the heater MH1 is heated to the first temperature range optimized for the measurement of the H₂ gas concentration during the measurement of H₂ gas, while the heater MH1 is heated to the second temperature range optimized for the measurement of the CO₂gas concentration during the measurement of CO₂ gas, allowing the concentrations of both H₂ gas and CO₂ gas in the measurement atmosphere to be measured with high sensitivity. In addition, in this fourth operation mode, when the level of the gas detection signal Vgas is higher than the threshold value V1 and lower than the threshold value V2, both the calculation of the gas concentration and the generation of the output signal Vout are stopped. Thus, when the target gas is switched from H₂ gas to CO₂ gas, the output signal Vout indicating the CO₂ gas concentration can be output in a state where the CO₂ gas concentration is sufficiently high, and when the target gas is switched from CO₂ gas to H₂ gas, the output signal Vout indicating the H₂ gas concentration can be output in a state where the H₂gas concentration is sufficiently high.

### Second Embodiment

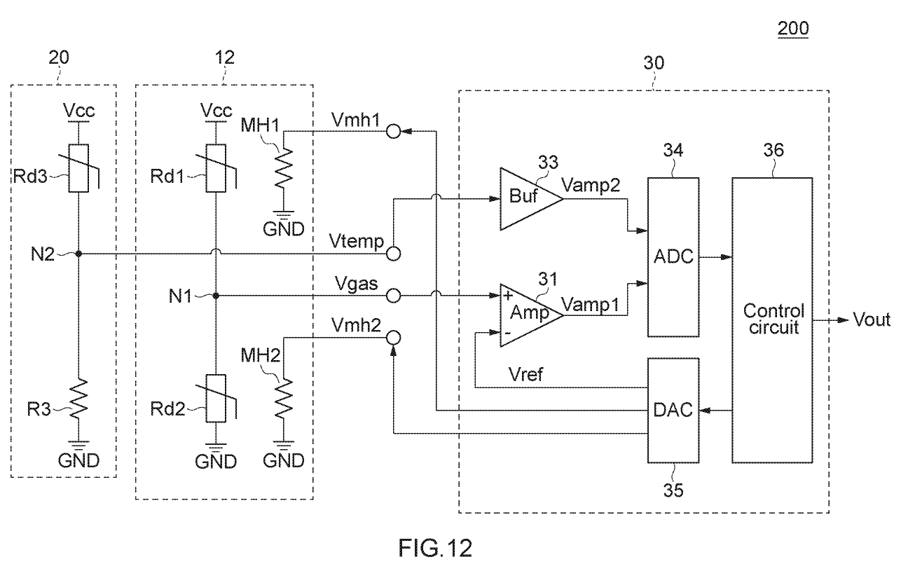

FIG. 12 is a circuit diagram illustrating the configuration of a gas sensor 200 according to a second embodiment of the technology described herein.

As illustrated in FIG. 12, the gas sensor 200 according to the second embodiment differs from the gas sensor 100 according to the first embodiment in that the sensor part 11 is replaced with a sensor part 12. Other basic configurations are the same as those of the gas sensor 100 according to the first embodiment, so the same reference numerals are given to the same elements, and overlapping description will be omitted.

The sensor part 12 includes thermistors Rd1 and Rd2, which are connected in series in this order between the power supply Vcc and the ground GND, and heaters MH1 and MH2. The thermistor Rd1 serves as a temperature-sensing element on the sensing side, and the thermistor Rd2 serves as a temperature-sensing element on the reference side. The thermistor Rd1 varies in temperature in response to a change in the temperature of the heater MH1, and the thermistor Rd2 varies in temperature in response to a change in the temperature of the heater MH2. The gas detection signal Vgas output from the sensor part 12 appears at a node N1 between the thermistors Rd1 and Rd2. In the second embodiment, the gas detection signal Vgas is an output signal derived from the thermistors Rd1 and Rd2. Like the thermistor Rd1, the thermistor Rd2 is a resistor whose resistance varies with temperature. The thermistor Rd2 may be made of the same material as the thermistor Rd1.

The heater MH2 is heated to a third temperature range by application of a heater voltage Vmh2 when the H₂gas concentration is measured, and to a fourth temperature range when the CO₂ gas concentration is measured. The third temperature range is a range in which the difference between the thermal conductivity of air and that of H₂ gas is small or negligible. The absolute value of the difference between the thermal conductivity of air and that of H₂ gas in the third temperature range is smaller than the absolute value of the difference between the thermal conductivity of air and that of H₂ gas in the first temperature range. The fourth temperature range is a range in which the difference between the thermal conductivity of air and that of CO₂ gas is small or negligible. The absolute value of the difference between the thermal conductivity of air and that of CO₂ gas in the fourth temperature range is smaller than the absolute value of the difference between the thermal conductivity of air and that of CO₂ gas in the second temperature range. Therefore, even if H₂ gas is present in the measurement atmosphere while the heater MH2 is maintained at the third temperature range, the heat dissipation characteristics of the heater MH2 remain substantially unchanged with the H₂ gas concentration. Similarly, even if CO₂ gas is present in the measurement atmosphere while the heater MH2 is maintained at the fourth temperature range, the heat dissipation characteristics of the heater MH2 remain substantially unchanged with the CO₂ gas concentration. The absolute value of the difference between the thermal conductivity of air and that of CO₂ gas in the third temperature range is smaller than the absolute value of the difference between the thermal conductivity of air and that of CO₂ gas in the first temperature range. The absolute value of the difference between the thermal conductivity of air and that of H₂ gas in the fourth temperature range is smaller than the absolute value of the difference between the thermal conductivity of air and that of H₂ gas in the second temperature range.

The third temperature range is different from the first and second temperature ranges. The third temperature range may be higher than the first temperature range. The fourth temperature range is different from the first and second temperature ranges. The fourth temperature range may be higher than the second temperature range. The third and fourth temperature ranges may be the same as or different from each other. The third temperature range is a predetermined temperature range included within a range of 410° C. or higher and 500° C. or lower, for example, a temperature range around 450° C.. The fourth temperature range is a predetermined temperature range included within a range of 250° C. or higher and 400°C or lower, for example, a temperature range around 300° C..

When the H₂ gas concentration is measured in the present embodiment, the heaters MH1 and MH2 are heated to the first temperature range and the third temperature range, respectively, for example. As described above, when H₂gas is present in the measurement atmosphere while the heater MH1 is maintained at the first temperature range, the heat dissipation characteristics of the heater MH1 increase as the H₂ gas concentration increases, so that the temperature of the heater MH1 decreases and, consequently, the temperature of the thermistor Rd1 also decreases. On the other hand, even if H₂ gas is present in the measurement atmosphere while the heater MH2 is maintained at the third temperature range, the heat dissipation characteristics of the heater MH1 remain substantially unchanged with the H₂ gas concentration, and thus the temperature of the thermistor Rd2 also remain substantially unchanged. Therefore, when H₂ gas is present in the measurement atmosphere while the heaters MH1 and MH2 are maintained at the first temperature range and at the third temperature range, the level of the gas detection signal Vgas decreases as the H₂ gas concentration increases.

On the other hand, even when another gas whose heat dissipation characteristics exhibit no significant difference between when the heater MH1 is heated to the first temperature range and when the heater MH2 is heated to the third temperature range is present in the measurement atmosphere, the concentration of this gas has little influence on the level of the gas detection signal Vgas. Thus, by heating the heaters MH1 and MH2 to the first temperature range and to the third temperature range, respectively, the H₂ gas concentration can be selectively detected.

When the CO₂ gas concentration is measured in the present embodiment, the heaters MH1 and MH2 are heated to the second temperature range and the fourth temperature range, respectively, for example. As described above, when CO₂gas is present in the measurement atmosphere while the heater MH1 is maintained at the second temperature range, the heat dissipation characteristics of the heater MH1 decrease as the CO₂ gas concentration increases, so that the temperature of the heater MH1 increases and, consequently, the temperature of the thermistor Rd1 also increases. On the other hand, even if CO₂ gas is present in the measurement atmosphere while the heater MH2 is maintained at the fourth temperature range, the heat dissipation characteristics of the heater MH2 remain substantially unchanged with the CO₂ gas concentration, and thus the temperature of the thermistor Rd2 also remains substantially unchanged. Therefore, when CO₂ gas is present in the measurement atmosphere while the heaters MH1 and MH2 are maintained at the second temperature range and at the fourth temperature range, respectively, the level of the gas detection signal Vgas increases as the CO₂ gas concentration increases.

On the other hand, even when another gas whose heat dissipation characteristics exhibit no significant difference between when the heater MH1 is heated to the second temperature range and when the heater MH2 is heated to the fourth temperature range is present in the measurement atmosphere, the concentration of this gas has little influence on the level of the gas detection signal Vgas. Thus, by heating the heaters MH1 and MH2 to the second temperature range and to the fourth temperature range, respectively, the CO₂ gas concentration can be selectively detected.

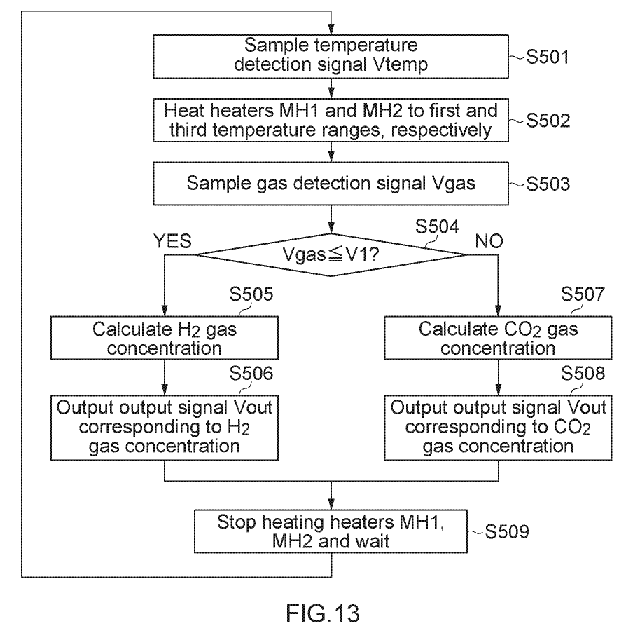

FIG. 13 is a flowchart illustrating a first operation mode of the gas sensor 200. The first operation mode of the gas sensor 200 is a mode based on measuring the H₂ gas concentration in the measurement atmosphere by heating the heaters MH1 and MH2 to the first and third temperature ranges, respectively. In this mode, when the CO₂ gas concentration in the measurement atmosphere increases in a state where the H₂ gas concentration in the measurement atmosphere is sufficiently low, the target gas is automatically switched from H₂ gas to CO₂ gas.

First, in the first operation mode of the gas sensor 200, the signal processing circuit 30 samples the temperature detection signal Vtemp to calculate the ambient temperature (step S501). Subsequently, the control circuit 36 included in the signal processing circuit 30 outputs a heater instruction value calculated based on the ambient temperature to the DA converter 35 to heat the heaters MH1 and MH2 to the first and third temperature ranges, respectively (step S502). The heater instruction value is converted into the heater voltages Vmh1 and Vmh2 by the DA converter 35, which are then applied to the heaters MH1 and MH2, respectively. Then, the signal processing circuit 30 samples the gas detection signal Vgas while the heaters MH1 and MH2 are maintained at the first and third temperature ranges, respectively (step S503).

The following operation is the same as the operation performed in the first mode of the gas sensor 100 described using FIG. 2. That is, it is determined whether the level of the gas detection signal Vgas is equal to or lower than the threshold value V1 (step S504); when the condition that the level of the gas detection signal Vgas is equal to or lower than the threshold value V1 is satisfied (Vgas ≤ V1), the H₂ gas concentration is calculated based on the gas detection signal Vgas (step S505); and the output signal Vout corresponding to the H₂ gas concentration is output to the outside (step S506). Thereafter, the heating of the heaters MH1 and MH2 is stopped, and waiting is performed until the next measurement timing (step S509). When the next measurement timing arrives, the flow returns to step S501 for repetition of the above operation.

On the other hand, when the condition that the level of the gas detection signal Vgas is equal to or lower than the threshold value V1 is not satisfied (Vgas > V1), the CO₂ gas concentration is calculated based on the gas detection signal Vgas (step S507), and the output signal Vout corresponding to the CO₂gas concentration is output to the outside (step S508). Thereafter, the heating of the heaters MH1 and MH2 is stopped, and waiting is performed until the next measurement timing (step S509). When the next measurement timing arrives, the flow returns to step S501 for repetition of the above operation.

In the first mode of the gas sensor 200, the concentrations of H₂ gas and CO₂ gas, the gas detection signal Vgas, and the output signal Vout change in the same manner as in the example illustrated in FIGS. 3A to 3C.

As described above, in the first operation mode of the gas sensor 200, similarly to the first operation mode of the gas sensor 100, the H₂ gas concentration in the measurement atmosphere is primarily measured, and when the CO₂ gas concentration in the measurement atmosphere increases, the target gas is automatically switched from H₂gas to CO₂ gas. Then, when the CO₂ gas concentration in the measurement atmosphere decreases, the target gas is switched from CO₂ gas to H₂ gas. In addition, in the first operation mode of the gas sensor 200, the heater MH1 is heated to the first temperature range optimized for the measurement of the H₂gas concentration, and the heater MH2 is heated to the third temperature range in which a change in the heat dissipation characteristics in response to a change in the H₂ gas concentration is small, allowing the H₂ gas concentration in the measurement atmosphere to be measured with high sensitivity and high accuracy.

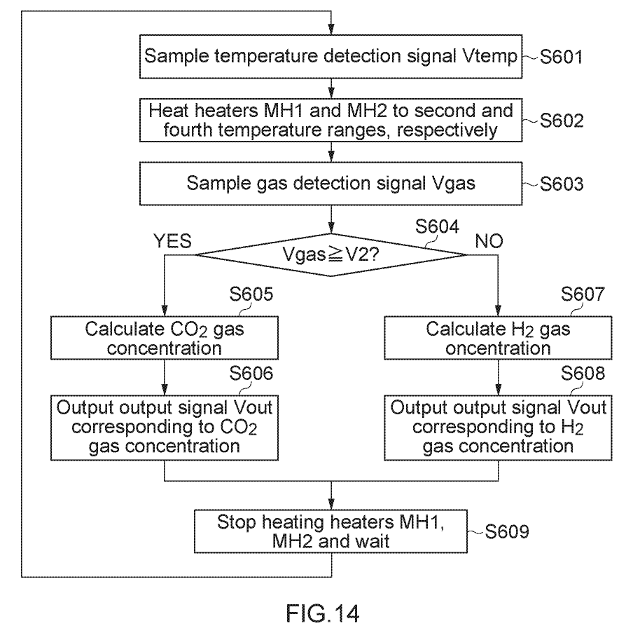

FIG. 14 is a flowchart illustrating a second operation mode of the gas sensor 200. The second operation mode of the gas sensor 200 is a mode based on measuring the CO₂ gas concentration in the measurement atmosphere by heating the heaters MH1 and MH2 to the second and fourth temperature ranges, respectively. In this mode, when the H₂ gas concentration in the measurement atmosphere increases in a state where the CO₂ gas concentration in the measurement atmosphere is sufficiently low, the target gas is automatically switched from CO₂ gas to H₂ gas.

First, in the second operation mode of the gas sensor 200, the signal processing circuit 30 samples the temperature detection signal Vtemp to calculate the ambient temperature (step S601). Subsequently, the control circuit 36 included in the signal processing circuit 30 outputs a heater instruction value calculated based on the ambient temperature to the DA converter 35 to heat the heaters MH1 and MH2 to the second and fourth temperature ranges, respectively (step S602). The heater instruction value is converted into the heater voltages Vmh1 and Vmh2 by the DA converter 35, which are then applied to the heaters MH1 and MH2, respectively. Then, the signal processing circuit 30 samples the gas detection signal Vgas while the heaters MH1 and MH2 are maintained at the second and fourth temperature ranges, respectively (step S603).

The following operation is the same as the operation performed in the second operation mode of the gas sensor 100 described using FIG. 4. That is, it is determined whether the level of the gas detection signal Vgas is equal to or higher than the threshold value V2 (step S604); when the condition that the level of the gas detection signal Vgas is equal to or higher than the threshold value V2 is satisfied (Vgas ≥ V2), the CO₂ gas concentration is calculated based on the gas detection signal Vgas (step S605); and the output signal Vout corresponding to the CO₂ gas concentration is output to the outside (step S606). Thereafter, the heating of the heaters MH1 and MH2 is stopped, and waiting is performed until the next measurement timing (step S609). When the next measurement timing arrives, the flow returns to step S601 for repetition of the above operation.

On the other hand, when the condition that the level of the gas detection signal Vgas is equal to or higher than the threshold value V2 is not satisfied (Vgas < V2), the H₂ gas concentration is calculated based on the gas detection signal Vgas (step S607), and the output signal Vout corresponding to the H₂ gas concentration is output to the outside (step S608). Thereafter, the heating of the heaters MH1 and MH2 is stopped, and waiting is performed until the next measurement timing (step S609). When the next measurement timing arrives, the flow returns to step S601 for repetition of the above operation.

In the second operation mode of the gas sensor 200, the concentrations of H₂ gas and CO₂ gas, the gas detection signal Vgas, and the output signal Vout change in the same manner as in the example illustrated in FIGS. 5A to 5C.

As described above, in the second operation mode of the gas sensor 200, similarly to the second operation mode of the gas sensor 100, the CO₂ gas concentration in the measurement atmosphere is primarily measured, and when the H₂ gas concentration in the measurement atmosphere increases, the target gas is automatically switched from CO₂ gas to H₂ gas. Then, when the H₂ gas concentration in the measurement atmosphere decreases, the target gas is switched from H₂ gas to CO₂ gas. In addition, in the second operation mode of the gas sensor 200, the heater MH1 is heated to the second temperature range optimized for the measurement of the CO₂ gas concentration, and the heater MH2 is heated to the fourth temperature range in which a change in the heat dissipation characteristics in response to a change in the CO₂ gas concentration is small, allowing the CO₂ gas concentration in the measurement atmosphere to be measured with high sensitivity and high accuracy.

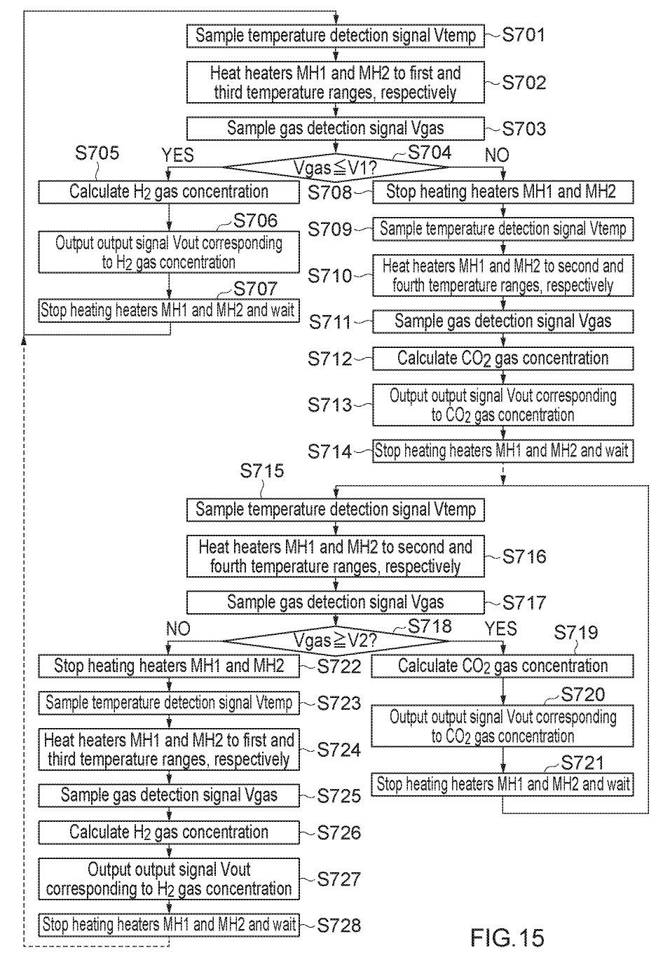

FIG. 15 is a flowchart illustrating a third operation mode of the gas sensor 200. The third operation mode of the gas sensor 200 is a mode in which the heaters MH1 and MH2 are heated to the first and third temperature ranges, respectively, when the H₂ gas concentration in the measurement atmosphere is measured, and to the second and fourth temperature ranges, respectively, when the CO₂ gas concentration in the measurement atmosphere is measured.

First, in the third operation mode of the gas sensor 200, when the H₂ gas concentration in the measurement atmosphere is measured, the signal processing circuit 30 samples the temperature detection signal Vtemp to calculate the ambient temperature (step S701). Subsequently, the control circuit 36 included in the signal processing circuit 30 outputs a heater instruction value calculated based on the ambient temperature to the DA converter 35 to heat the heaters MH1 and MH2 to the first and third temperature ranges, respectively (step S702). The heater instruction value is converted into the heater voltages Vmh1 and Vmh2 by the DA converter 35, which are then applied to the heaters MH1 and MH2, respectively. Then, the signal processing circuit 30 samples the gas detection signal Vgas while the heaters MH1 and MH2 are maintained at the first and third temperature ranges, respectively (step S703).

Then, the signal processing circuit 30 determines whether the level of the gas detection signal Vgas is equal to or lower than the threshold value V1 (step S704). The operation performed when the condition that the level of the gas detection signal Vgas is equal to or lower than the threshold value V1 is satisfied (Vgas ≤ V1) is the same as that performed in the third operation mode of the gas sensor 100 described using FIG. 6. That is, the H₂ gas concentration is calculated based on the gas detection signal Vgas (step S705), and the output signal Vout corresponding to the H₂ gas concentration is output to the outside (step S706). Thereafter, the heating of the heaters MH1 and MH2 is stopped, and waiting is performed until the next measurement timing (step S707). When the next measurement timing arrives, the flow returns to step S701 for repetition of the above operation.

On the other hand, when the condition that the level of the gas detection signal Vgas is equal to or lower than the threshold value V1 is not satisfied (Vgas > V1), the heating of the heaters MH1 and MH2 is temporarily stopped (step S708), and the signal processing circuit 30 samples the temperature detection signal Vtemp again to calculate the ambient temperature (step S709). Subsequently, the control circuit 36 included in the signal processing circuit 30 outputs a heater instruction value calculated based on the ambient temperature to the DA converter 35 to heat the heaters MH1 and MH2 to the second and fourth temperature ranges, respectively (step S710). The heater instruction value is converted into the heater voltages Vmh1 and Vmh2 by the DA converter 35, which are then applied to the heaters MH1 and MH2, respectively. Then, the signal processing circuit 30 samples the gas detection signal Vgas while the heaters MH1 and MH2 are maintained at the second and fourth temperature ranges, respectively (step S711).

Then, the CO₂ gas concentration is calculated based on the gas detection signal Vgas (step S712), and the output signal Vout corresponding to the CO₂gas concentration is output to the outside (step S713). Thereafter, the heating of the heaters MH1 and MH2 is stopped, and waiting is performed until the next measurement timing (step S714).

When the next measurement timing arrives, the signal processing circuit 30 samples the temperature detection signal Vtemp again to calculate the ambient temperature (step S715). Subsequently, the control circuit 36 included in the signal processing circuit 30 outputs a heater instruction value calculated based on the ambient temperature to the DA converter 35 to heat the heaters MH1 and MH2 to the second and fourth temperature ranges, respectively (step S716). The heater instruction value is converted into the heater voltages Vmh1 and Vmh2 by the DA converter 35, which are then applied to the heaters MH1 and MH2, respectively. Then, the signal processing circuit 30 samples the gas detection signal Vgas while the heaters MH1 and MH2 are maintained at the second and fourth temperature ranges, respectively (step S717).

Then, the signal processing circuit 30 determines whether the level of the gas detection signal Vgas is equal to or higher than the threshold value V2 (step S718). The operation performed when the condition that the level of the gas detection signal Vgas is equal to or higher than the threshold value V2 is satisfied (Vgas ≥ V2) is the same as that performed in the third operation mode of the gas sensor 100 described using FIG. 6. That is, the CO₂ gas concentration is calculated based on the gas detection signal Vgas (step S719), and the output signal Vout corresponding to the CO₂ gas concentration is output to the outside (step S720). Thereafter, the heating of the heaters MH1 and MH2 is stopped, and waiting is performed until the next measurement timing (step S721). When the next measurement timing arrives, the flow returns to step S715 for repetition of the above operation.

On the other hand, when the condition that the level of the gas detection signal Vgas is equal to or higher than the threshold value V2 is not satisfied (Vgas < V2), the heating of the heaters MH1 and MH2 is temporarily stopped (step S722), and the signal processing circuit 30 samples the temperature detection signal Vtemp again to calculate the ambient temperature (step S723). Subsequently, the control circuit 36 included in the signal processing circuit 30 outputs a heater instruction value calculated based on the ambient temperature to the DA converter 35 to heat the heaters MH1 and MH2 to the first and third temperature ranges, respectively (step S724). The heater instruction value is converted into the heater voltages Vmh1 and Vmh2 by the DA converter 35, which are then applied to the heaters MH1 and MH2, respectively. Then, the signal processing circuit 30 samples the gas detection signal Vgas while the heaters MH1 and MH2 are maintained at the first and second temperature ranges (step S725).

Then, the H₂ gas concentration is calculated based on the gas detection signal Vgas (step S726), and the output signal Vout corresponding to the H₂ gas concentration is output to the outside (step S727). Thereafter, the heating of the heaters MH1 and MH2 is stopped, and waiting is performed until the next measurement timing (step S728). When the next measurement timing arrives, the flow returns to step S701 for repetition of the above operation.

In the third operation mode of the gas sensor 200, the concentrations of H₂ gas and CO₂ gas, the gas detection signal Vgas, and the output signal Vout change in the same manner as in the example illustrated in FIGS. 7A to 7C and 8A to 8C.

As described above, in the third operation mode of the gas sensor 200, when the CO₂gas concentration in the measurement atmosphere increases during the measurement of the H₂ gas concentration, the target gas is automatically switched from H₂ gas to CO₂ gas; when the H₂ gas concentration in the measurement atmosphere increases during the measurement of the CO₂ gas concentration, the target gas is automatically switched from CO₂ gas to H₂ gas. In the third operation mode of the gas sensor 200, during the measurement of H₂ gas, the heater MH1 is heated to the first temperature range optimized for the measurement of the H₂ gas concentration, and the heater MH2 is heated to the third temperature range in which a change in the heat dissipation characteristics in response to a change in the H₂ gas concentration is small, allowing the H₂ gas concentration in the measurement atmosphere to be measured with high sensitivity and high accuracy. Similarly, during the measurement of CO₂ gas, the heater MH1 is heated to the second temperature range optimized for the measurement of the CO₂ gas concentration, and the heater MH2 is heated to the fourth temperature range in which a change in the heat dissipation characteristics in response to a change in the CO₂ gas concentration is small, allowing the CO₂ gas concentration in the measurement atmosphere to be measured with high sensitivity and high accuracy. Although, in the above example, the flow proceeds to step S715 when the next measurement timing arrives in step S714, the flow may proceed to step S701 in this case.

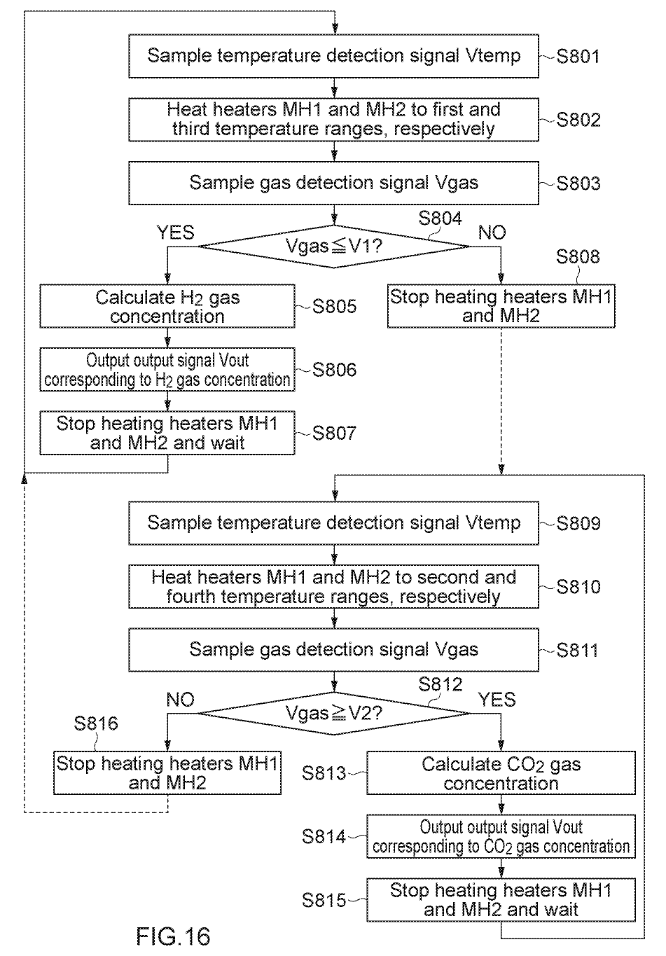

FIG. 16 is a flowchart illustrating a fourth operation mode of the gas sensor 200. In the fourth operation mode of the gas sensor 200, when the H₂ gas concentration in the measurement atmosphere is measured, the heaters MH1 and MH2 are heated to the first and third temperature ranges, respectively; whereas when the CO₂ gas concentration in the measurement atmosphere is measured, the heaters MH1 and MH2 are heated to the second and fourth temperature ranges, respectively, and a determination is made using the threshold value after the switching of the target gas and before the measurement of the concentration of the target gas.

First, in the fourth operation mode of the gas sensor 200, when the H₂ gas concentration in the measurement atmosphere is measured, the signal processing circuit 30 samples the temperature detection signal Vtemp to calculate the ambient temperature (step S801). Subsequently, the control circuit 36 included in the signal processing circuit 30 outputs a heater instruction value calculated based on the ambient temperature to the DA converter 35 to heat the heaters MH1 and MH2 to the first and third temperature ranges, respectively (step S802). The heater instruction value is converted into the heater voltages Vmh1 and Vmh2 by the DA converter 35, which are then applied to the heaters MH1 and MH2, respectively. Then, the signal processing circuit 30 samples the gas detection signal Vgas while the heaters MH1 and MH2 are maintained at the first and third temperature ranges, respectively (step S803).

Then, the signal processing circuit 30 determines whether the level of the gas detection signal Vgas is equal to or lower than the threshold value V1 (step S804). The operation performed when the condition that the level of the gas detection signal Vgas is equal to or lower than the threshold value V1 is satisfied (Vgas ≤ V1) is the same as that performed in the fourth operation mode of the gas sensor 100 described using FIG. 9. That is, the H₂ gas concentration is calculated based on the gas detection signal Vgas (step S805), and the output signal Vout corresponding to the H₂ gas concentration is output to the outside (step S806). Thereafter, the heating of the heaters MH1 and MH2 is stopped, and waiting is performed until the next measurement timing (step S807). When the next measurement timing arrives, the flow returns to step S801 for repetition of the above operation.

On the other hand, when the condition that the level of the gas detection signal Vgas is equal to or lower than the threshold value V1 is not satisfied (Vgas > V1), the heating of the heaters MH1 and MH2 is temporarily stopped (step S808), and the target gas is switched from H₂ gas to CO₂ gas.

When the target gas is switched from H₂ gas to CO₂ gas, the signal processing circuit 30 samples the temperature detection signal Vtemp to calculate the ambient temperature (step S809). Subsequently, the control circuit 36 included in the signal processing circuit 30 outputs a heater instruction value calculated based on the ambient temperature to the DA converter 35 to heat the heaters MH1 and MH2 to the second and fourth temperature ranges, respectively (step S810). The heater instruction value is converted into the heater voltages Vmh1 and Vmh2 by the DA converter 35, which are applied to the heaters MH1 and MH2, respectively. Then, the signal processing circuit 30 samples the gas detection signal Vgas while the heaters MH1 and MH2 are maintained at the second and fourth temperature ranges, respectively (step S811).

Then, the signal processing circuit 30 determines whether the level of the gas detection signal Vgas is equal to or higher than the threshold value V2 (step S812). The operation performed when the condition that the level of the gas detection signal Vgas is equal to or higher than the threshold value V2 is satisfied (Vgas ≥ V1) is the same as that performed in the fourth operation mode of the gas sensor 100 described using FIG. 9. That is, the CO₂ gas concentration is calculated based on the gas detection signal Vgas (step S813), and the output signal Vout corresponding to the CO₂ gas concentration is output to the outside (step S814). Thereafter, the heating of the heaters MH1 and MH2 is stopped, and waiting is performed until the next measurement timing (step S815). When the next measurement timing arrives, the flow returns to step S809 for repetition of the above operation.

On the other hand, when the condition that the level of the gas detection signal Vgas is equal to or higher than the threshold value V2 is not satisfied (Vgas < V2), the heating of the heaters MH1 and MH2 is temporarily stopped (step S816), and the flow returns to step S801, where the target gas is switched from CO₂ gas to H₂gas.

In the fourth mode of the gas sensor 200, the concentrations of H₂ gas and CO₂ gas, the gas detection signal Vgas, and the output signal Vout change in the same manner as in the example illustrated in FIGS. 10A to 10C and 11A to 11C.

As described above, in the fourth operation mode of the gas sensor 200, the heater MH1 is heated to the first temperature range optimized for the measurement of the H₂ gas concentration during the measurement of H₂ gas, and the heater MH2 is heated to the third temperature range in which a change in the heat dissipation characteristics in response to a change in the H₂ gas concentration is small, allowing the H₂ gas concentration in the measurement atmosphere to be measured with high sensitivity and high accuracy. Similarly, the heater MH1 is heated to the second temperature range optimized for the measurement of the CO₂ gas concentration during the measurement of CO₂ gas concentration, and the heater MH2 is heated to the fourth temperature range in which a change in the heat dissipation characteristics in response to a change in the CO₂ gas concentration is small, allowing the CO₂ gas concentration in the measurement atmosphere to be measured with high sensitivity and high accuracy.

In addition, in the fourth operation mode of the gas sensor 200, when the level of the gas detection signal Vgas is higher than the threshold value V1 and lower than the threshold value V2, both the calculation of the gas concentration and the generation of the output signal Vout are stopped. Thus, similarly to the fourth operation mode of the gas sensor 100, when the target gas is switched from H₂ gas to CO₂ gas, the output signal Vout indicating the CO₂ gas concentration can be output in a state where the CO₂ gas concentration is sufficiently high, and when the target gas is switched from CO₂ gas to H₂ gas, the output signal Vout indicating the H₂ gas concentration can be output in a state where the H₂ gas concentration is sufficiently high.

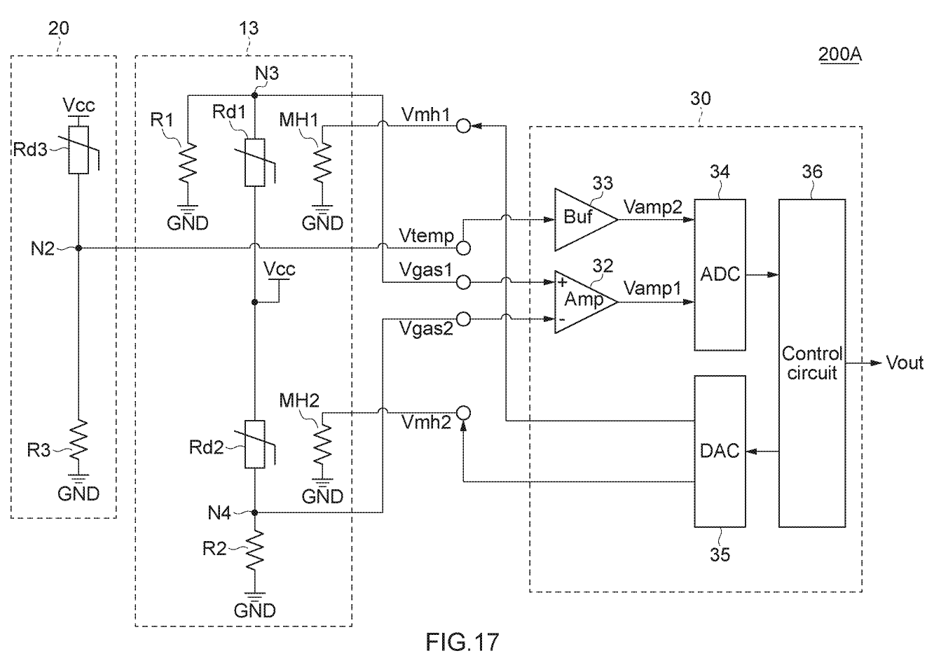

FIG. 17 is a circuit diagram illustrating the configuration of a gas sensor 200A according to a first modification of the second embodiment.

As illustrated in FIG. 17, the gas sensor 200A according to the first modification of the second embodiment differs from the gas sensor 200 according to the second embodiment in that the sensor part 12 is replaced with a sensor part 13 and that the differential amplifier 31 included in the signal processing circuit 30 is replaced with a differential amplifier 32. Other basic configurations are the same as those of the gas sensor 200 according to the second embodiment, so the same reference numerals are given to the same elements, and overlapping description will be omitted.

The sensor part 13 includes the thermistor Rd1 and a fixed resistor R1, which are connected in series in this order between the power supply Vcc and the ground GND, the thermistor Rd2 and a fixed resistor R2, which are connected in series in this order between the power supply Vcc and the ground GND, and the heaters MH1 and MH2. The thermistor Rd1 is a temperature-sensing element whose temperature varies in response to a change in the temperature of the heater MH1. The thermistor Rd2 is a temperature-sensing element whose temperature varies in response to a change in the temperature of the heater MH2. A gas detection signal Vgas1 appears at a node N3 between the thermistor Rd1 and the fixed resistor R1. A gas detection signal Vgas2 appears at a node N4 between the thermistor Rd2 and the fixed resistor R2.

The differential amplifier 32 included in the signal processing circuit 30 compares the gas detection signal Vgas1 and the gas detection signal Vgas2 to generate an amplification signal Vamp1 corresponding to the amplified level difference (= Vgas1 – Vgas2) between the gas detection signal Vgas1 and the gas detection signal Vgas2.

As exemplified by the gas sensor 200A according to the first modification of the second embodiment, the thermistors Rd1 and Rd2 need not be connected in series between the power supply Vcc and the ground GND but may be connected in parallel therebetween to calculate the concentration of a target gas based on the difference between the output voltage (gas detection signal Vgas1) derived from the thermistor Rd1 and the output voltage (gas detection signal Vgas2) derived from the thermistor Rd2.

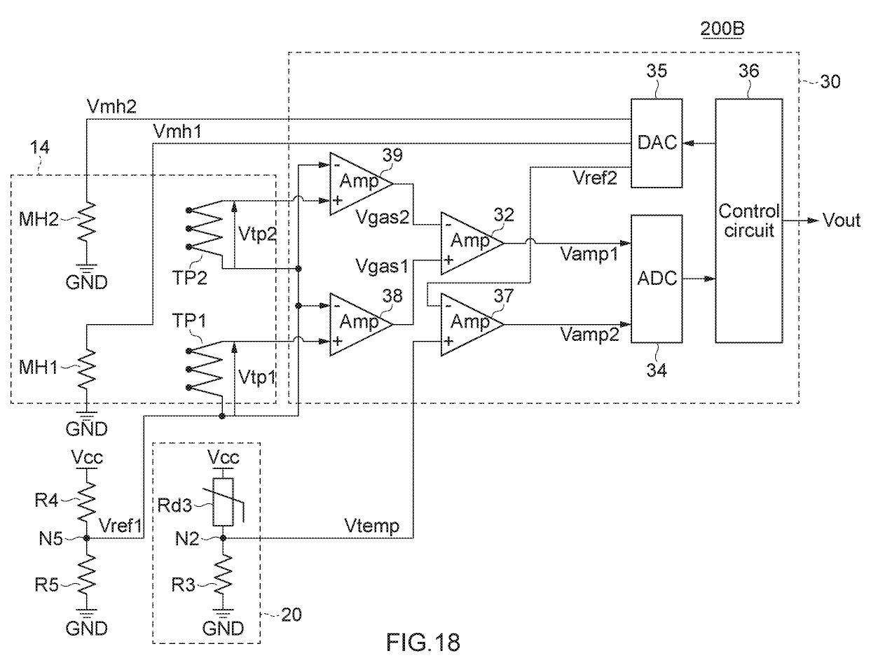

FIG. 18 is a circuit diagram illustrating the configuration of a gas sensor 200B according to a second modification of the second embodiment.

As illustrated in FIG. 18, the gas sensor 200B according to the second modification of the second embodiment differs from the gas sensor 200A according to the first modification of the second embodiment in that the sensor part 13 is replaced with a sensor part 14 and that differential amplifiers 37 to 39 are provided in the signal processing circuit 30. Other basic configurations are the same as those of the gas sensor 200A according to the first modification of the second embodiment, so the same reference numerals are given to the same elements, and overlapping description will be omitted.

The sensor part 14 includes thermopile elements TP1, TP2 and the heaters MH1, MH2. The hot junction of the thermopile element TP1 varies in temperature in response to a change in the temperature of the heater MH1, and the hot junction of the thermopile element TP2 varies in temperature in response to a change in the temperature of the heater MH2. The thermopile elements TP1 and TP2 are each an element in which a potential difference appearing between both ends thereof varies depending on temperature. The potential difference appearing between both ends of the thermopile element TP1 is used as an output signal Vtp1, and the potential difference appearing between both ends of the thermopile element TP2 is used as an output signal Vtp2. A reference potential Vref1 is generated by fixed resistors R4 and R5. The fixed resistors R4 and R5 are connected in series between the power supply Vcc and the ground GND, and the reference potential Vref1 appears at a node N5 between the fixed resistors R4 and R5. The reference potential Vref1 and the output signals Vtp1 and Vtp2 are supplied to the signal processing circuit 30. The potential supplied to the non-inversion input terminal (+) of the differential amplifier 38 has a level obtained by superimposing the output signal Vtp1 corresponding to the temperature-dependent electromotive force of the thermopile element TP1 on the reference potential Vref1. The potential supplied to the non-inversion input terminal (+) of the differential amplifier 39 has a level obtained by superimposing the output signal Vtp2 corresponding to the temperature-dependent electromotive force of the thermopile element TP2 on the reference potential Vref1.

The output signal Vtp1 is amplified by the differential amplifier 38 included in the signal processing circuit 30 to generate the gas detection signal Vgas1. The differential amplifier 38 compares the reference potential Vref1 supplied to the inversion input terminal (-) thereof with the level of (Vref1 + Vtp1) supplied to the non-inversion input terminal (+) thereof to generate the gas detection signal Vgas1 corresponding to the amplified level difference (= Vtp1) between the reference potential Vref1 and (Vref1 + Vtp1).

The output signal Vtp2 is amplified by the differential amplifier 39 included in the signal processing circuit 30 to generate the gas detection signal Vgas2. The differential amplifier 39 compares the reference potential Vref1 supplied to the inversion input terminal (-) thereof with the level of (Vref1 + Vtp2) supplied to the non-inversion input terminal (+) thereof to generate the gas detection signal Vgas2 corresponding to the amplified level difference (= Vtp2) between the reference potential Vref1 and (Vref1 + Vtp2).

Similarly to the first modification of the second embodiment, the differential amplifier 32 compares the gas detection signal Vgas1 with the gas detection signal Vgas2 to generate an amplification signal Vamp1 corresponding to the amplified level difference (= Vgas1 – Vgas2) between the gas detection signal Vgas1 and the gas detection signal Vgas2.

In the present modification, the temperature detection signal Vtemp output from the temperature sensor 20 is supplied to the differential amplifier 37 included in the signal processing circuit 30. The differential amplifier 37 compares the temperature detection signal Vtemp with the reference potential Vref2 to generate an amplification signal Vamp2 corresponding to the amplified level difference (= Vtemp – Vref2) between the temperature detection signal Vtemp and the reference voltage Vref2. Alternatively, as in the gas sensor 100 according to the first embodiment and the gas sensor 200 according to the second embodiment, the amplification signal Vamp2 may be generated by buffering the temperature detection signal Vtemp using the buffer 33.

Even with such a circuit configuration, when the heater MH1 is heated to the first temperature range, an increase in the H₂ gas concentration in the measurement atmosphere causes the temperature of the heater MH1 to decrease, whereby the temperature at the hot junction of the thermopile element TP1 also decreases in accordance therewith, so that the level of the gas detection signal Vgas1 corresponding to the thermal electromotive force of the thermopile element TP1 decreases. On the other hand, when the heater MH2 is heated to the third temperature range, the temperature of the heater MH2 and the temperature at the hot junction of the thermopile element TP2 hardly increase even if the H₂ gas concentration in the measurement atmosphere increases, so that the level of the gas detection signal Vgas2 corresponding to the thermal electromotive force of the thermopile element TP2 hardly increases. Similarly, when the heater MH1 is heated to the second temperature range, an increase in the CO₂ gas concentration in the measurement atmosphere causes the temperature of the heater MH1 to increase, whereby the temperature at the hot junction of the thermopile element TP1 also increases in accordance therewith, so that the level of the gas detection signal Vgas1 corresponding to the thermal electromotive force of the thermopile element TP1 increases. On the other hand, when the heater MH2 is heated to the fourth temperature range, the temperature of the heater MH2 and the temperature at the hot junction of the thermopile element TP2 hardly increase even if the CO₂ gas concentration in the measurement atmosphere increases, so that the level of the gas detection signal Vgas2 corresponding to the thermal electromotive force of the thermopile element TP2 hardly increases. Such a level difference between the gas detection signals Vgas1 and Vgas2 is amplified by the differential amplifier 32 to generate the amplification signal Vamp1.

As exemplified by the gas sensor 200B according to the second modification of the second embodiment, it is not essential to use the thermistor as a temperature-sensing element; instead, other types of temperature-sensing elements, such as a thermopile element, may be employed.

### Third Embodiment

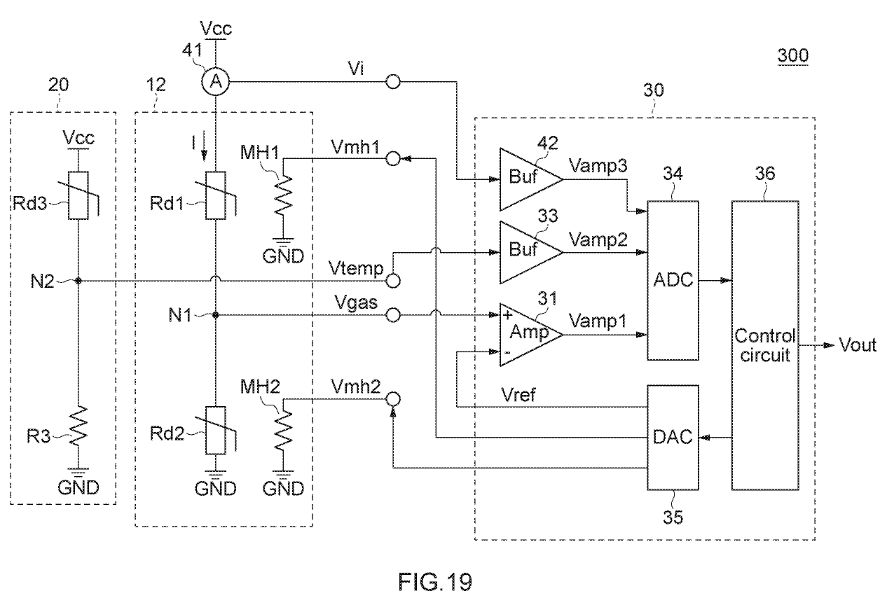

FIG. 19 is a circuit diagram illustrating a gas sensor 300 according to a third embodiment of the technology described herein.

As illustrated in FIG. 19, the gas sensor 300 according to the third embodiment differs from the gas sensor 200 according to the second embodiment in that a current sensor 41 is connected in series with the thermistors Rd1 and Rd2 and that a buffer 42 is additionally provided in the signal processing circuit 30. Other basic configurations are the same as those of the gas sensor 200 according to the second embodiment, so the same reference numerals are given to the same elements, and overlapping description will be omitted.

The current sensor 41 measures current flowing through the thermistors Rd1 and Rd2 to generate a current detection signal Vi. The buffer 42 buffers the current detection signal Vi to generate an amplification signal Vamp3. The amplification signal Vamp3 is converted into a corresponding digital value by the AD converter 34, which is then supplied to the control circuit 36.

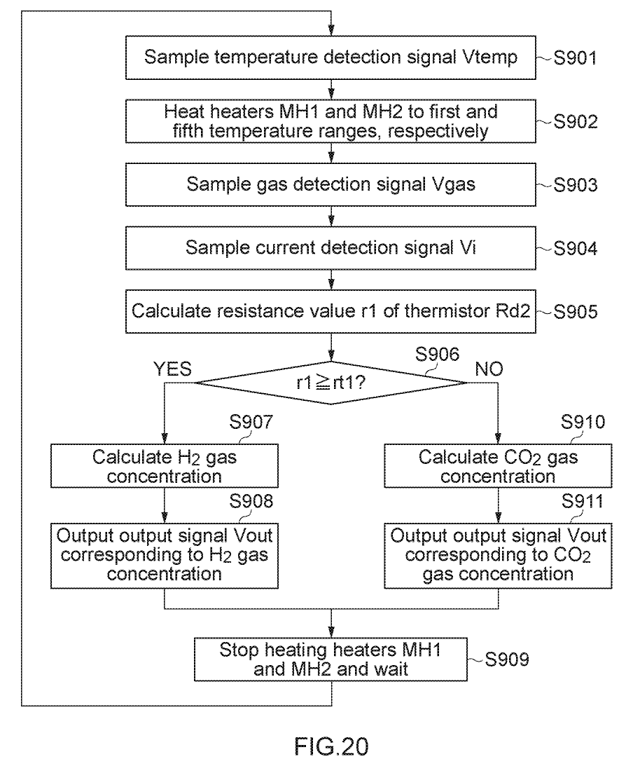

FIG. 20 is a flowchart illustrating a first operation mode of the gas sensor 300. The first operation mode of the gas sensor 300 is a mode based on measuring the H₂ gas concentration in the measurement atmosphere by heating the heaters MH1 and MH2 to the first temperature range and a fifth temperature range, respectively. In this mode, when the CO₂ gas concentration in the measurement atmosphere increases in a state where the H₂ gas concentration in the measurement atmosphere is sufficiently low, the target gas is automatically switched from H₂ gas to CO₂ gas.

The fifth temperature range is a range in which the difference between the thermal conductivity of air and that of H₂ gas is small or negligible. The absolute value of the difference between the thermal conductivity of air and that of H₂ gas in the fifth temperature range is smaller than the absolute value of the difference between the thermal conductivity of air and that of H₂ gas in the first temperature range. The fifth temperature range is a range in which the absolute value of the difference between the thermal conductivity of air and that of CO₂ gas is larger than the absolute value of the difference between the thermal conductivity of air and that of H₂ gas. In the fifth temperature range, the difference between the thermal conductivity of air and that of H₂ gas may be negligible. Therefore, even if H₂ gas is present in the measurement atmosphere while the heater MH2 is maintained at the fifth temperature range, the heat dissipation characteristics of the heater MH2 remain substantially unchanged with the H₂ gas concentration. Further, the absolute value of the difference between the thermal conductivity of air and that of CO₂ gas in the fifth temperature range is smaller than the absolute value of the difference between the thermal conductivity of air and that of CO₂ gas in the first temperature range. The fifth temperature range may be the same as the above-described third temperature range. The fifth temperature range is different from the first and second temperature ranges. The fifth temperature range may be higher than the first temperature range. The fifth temperature range is a predetermined temperature range included within a range of 410° C. or higher and 500° C. or lower, for example, a temperature range around 450° C..

First, in the first operation mode of the gas sensor 300, the signal processing circuit 30 samples the temperature detection signal Vtemp to calculate the ambient temperature (step S901). Subsequently, the control circuit 36 included in the signal processing circuit 30 outputs a heater instruction value calculated based on the ambient temperature to the DA converter 35 to heat the heaters MH1 and MH2 to the first and fifth temperature ranges, respectively (step S902). The heater instruction value is converted into the heater voltages Vmh1 and Vmh2 by the DA converter 35, which are then applied to the heaters MH1 and MH2, respectively. Then, the signal processing circuit 30 samples the gas detection signal Vgas while the heaters MH1 and MH2 are maintained at the first and fifth temperature ranges, respectively (step S903).

Then, the signal processing circuit 30 samples the current detection signal Vi (step S904). Subsequently, the control circuit 36 included in the signal processing circuit 30 calculates a resistance value r1 of the thermistor Rd2 based on the digital value of the amplification signal Vamp3 (step S905). Assuming that the value of a current flowing through the thermistor Rd2 is I, the resistance value r1 of the thermistor Rd2 can be calculated by the following equation: r1 = Vgas/I. The gas detection signal Vgas, the current detection signal Vi, and the amplification signal Vamp3 are signals indicating the resistance value r1 of the thermistor Rd2 and derived from the thermistor Rd2.

Then, the signal processing circuit 30 determines whether the resistance value r1 of the thermistor Rd2 is equal to or larger than a threshold value rt1 (step S906). This determination corresponds to determining whether the target gas is switched from H₂ gas to CO₂ gas. That is, in a state where the thermistor Rd2 is maintained at the fifth temperature range, the resistance value r1 of the thermistor Rd2 increases as the H₂ gas concentration in the measurement atmosphere increases, or the resistance value rt1 of the thermistor Rd2 remains almost unchanged with the H₂ gas concentration in the measurement atmosphere, so that the resistance value r1 of the thermistor Rd2 should be equal to or larger than the resistance value r1 that is to be obtained when the H₂ gas concentration is around zero. Therefore, when the condition that the resistance value r1 of the thermistor Rd2 is equal to or larger than the threshold value rt1 is satisfied (r1 ≥ rt1), the H₂ gas concentration is calculated based on the gas detection signal Vgas (step S907), and the output signal Vout corresponding to the H₂ gas concentration is output to the outside (step S908). Thereafter, the heating of the heaters MH1 and MH2 is stopped, and waiting is performed until the next measurement timing (step S909). When the next measurement timing arrives, the flow returns to step S901 for repetition of the above operation.

On the other hand, when the condition that the resistance value r1 of the thermistor Rd2 is equal to or larger than the threshold value rt1 is not satisfied (r1 < rt1), it is suggested that CO₂ gas is present at a high concentration in the measurement atmosphere. That is, when CO₂ gas is present in the measurement atmosphere while the thermistor Rd2 is maintained at the fifth temperature range, the heat dissipation characteristics of the heater MH2 decrease as the CO₂ gas concentration increases, so that the temperature of the heater MH2 increases and, consequently, the temperature of the thermistor Rd2 also increases. Thus, the resistance value r1 of the thermistor Rd2 decreases as the CO₂ gas concentration in the measurement atmosphere increases. In this case, the CO₂ gas concentration is calculated based on the gas detection signal Vgas (step S910), and the output signal Vout corresponding to the CO₂ gas concentration is output to the outside (step S911). Thereafter, the heating of the heaters MH1 and MH2 is stopped, and waiting is performed until the next measurement timing (step S909). When the next measurement timing arrives, the flow returns to step S901 for repetition of the above operation.

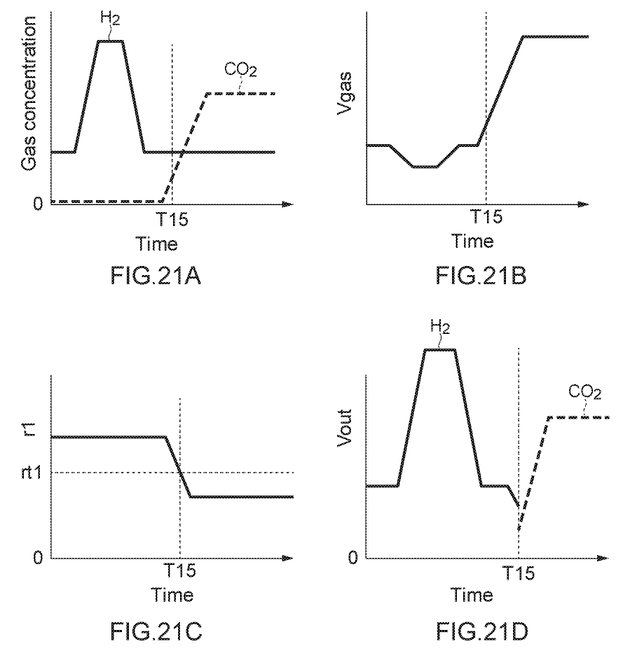

FIGS. 21A to 21D are graphs illustrating changes in the concentrations of H₂ gas and CO₂ gas, a change in the gas detection signal Vgas, a change in the resistance value r1 of the thermistor Rd2, and a change in the output signal Vout, respectively, in the first operation mode of the gas sensor 300.

In the example illustrated in FIG. 21A, after the H₂ gas concentration temporarily changes, the CO₂ gas concentration sharply increases around time T15. In this case, as illustrated in FIG. 21B, the level of the gas detection signal Vgas changes in association with the H₂ gas concentration until time T15 and sharply increases at time T15. The sharp increase in the level of the gas detection signal Vgas is caused by the sharp increase in the CO₂ gas concentration. Since the heat dissipation characteristics of the thermistor Rd2 decrease as the CO₂ gas concentration increases, the resistance value r1 of the thermistor Rd2 decreases and falls below the threshold value rt1 at time T15, as illustrated in FIG. 21C. As a result, as illustrated in FIG. 21D, the signal processing circuit 30 generates the output signal Vout indicating the H₂ gas concentration until time T15 and generates the output signal Vout indicating the CO₂ gas concentration after time T15.

As described above, in the first operation mode of the gas sensor 300, similarly to the first operation modes of the gas sensors 100 and 200, the H₂ gas concentration in the measurement atmosphere is primarily measured, and when the resistance value r1 of the thermistor Rd2 falls below the threshold value rt1 as a result of an increase in the CO₂ gas concentration in the measurement atmosphere, the target gas is automatically switched from H₂ gas to CO₂gas. Then, when the CO₂ gas concentration in the measurement atmosphere decreases, the target gas is switched from CO₂ gas to H₂ gas. Further, in the first operation mode of the gas sensor 300, an increase in the CO₂ gas concentration is detected by directly measuring the resistance value r1 of the thermistor Rd2, rather than the level of the gas detection signal Vgas, so that when the CO₂ gas concentration increases, the target gas can be rapidly switched from H₂ gas to CO₂ gas. Note that, when the resistance value r1 of the thermistor Rd2 is measured, heating of the heater MH1 may be omitted.

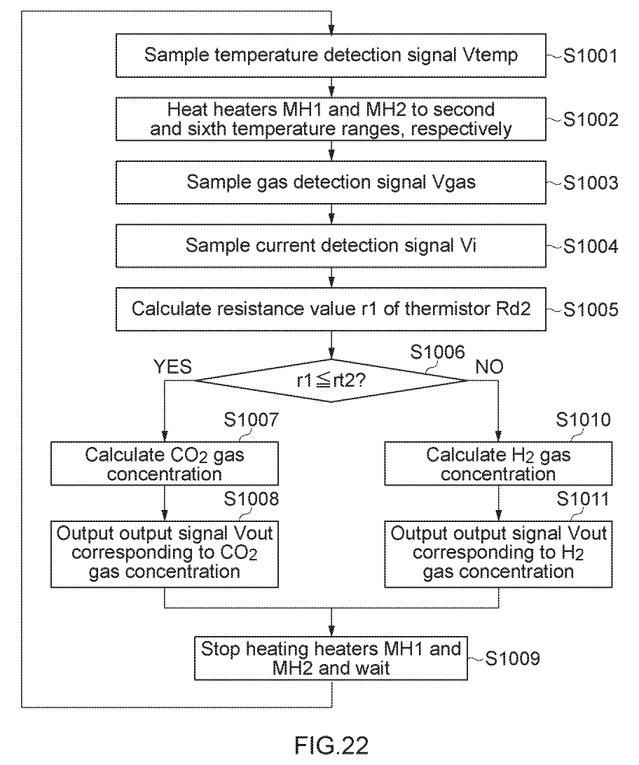

FIG. 22 is a flowchart illustrating a second operation mode of the gas sensor 300. The second operation mode of the gas sensor 300 is a mode based on measuring the CO₂ gas concentration in the measurement atmosphere by heating the heaters MH1 and MH2 to the second temperature range and a sixth temperature range, respectively. In this mode, when the H₂ gas concentration in the measurement atmosphere increases in a state where the CO₂ gas concentration in the measurement atmosphere is sufficiently low, the target gas is automatically switched from CO₂gas to H₂ gas.

The sixth temperature range is a range in which the difference between the thermal conductivity of air and that of CO₂ gas is small or negligible. The absolute value of the difference between the thermal conductivity of air and that of CO₂ gas in the sixth temperature range is smaller than the absolute value of the difference between the thermal conductivity of air and that of CO₂ gas in the second temperature range. The sixth temperature range is a range in which the absolute value of the difference between the thermal conductivity of air and that of H₂ gas is larger than the absolute value of the difference between the thermal conductivity of air and that of CO₂ gas. In the sixth temperature range, the difference between the thermal conductivity of air and that of CO₂ gas may be negligible. Therefore, even if CO₂ gas is present in the measurement atmosphere while the heater MH2 is maintained at the sixth temperature range, the heat dissipation characteristics of the heater MH2 remain substantially unchanged with the CO₂ gas concentration. Further, the absolute value of the difference between the thermal conductivity of air and that of H₂ gas in the sixth temperature range is smaller than the absolute value of the difference between the thermal conductivity of air and that of H₂ gas in the second temperature range. The sixth temperature range may be the same as the above-described fourth temperature range. The sixth temperature range is different from the first, second, and fifth temperature ranges. The sixth temperature range may be higher than the second temperature range. The sixth temperature range is a predetermined temperature range included within a range of 250° C. or higher and 400° C. or lower, for example, a temperature range around 300° C..

First, in the second operation mode of the gas sensor 300, the signal processing circuit 30 samples the temperature detection signal Vtemp to calculate the ambient temperature (step S1001). Subsequently, the control circuit 36 included in the signal processing circuit 30 outputs a heater instruction value calculated based on the ambient temperature to the DA converter 35 to heat the heaters MH1 and MH2 to the second and sixth temperature ranges, respectively (step S1002). The heater instruction value is converted into the heater voltages Vmh1 and Vmh2 by the DA converter 35, which are then applied to the heaters MH1 and MH2, respectively. Then, the signal processing circuit 30 samples the gas detection signal Vgas while the heaters MH1 and MH2 are maintained at the second and sixth temperature ranges, respectively (step S1003).

Then, the signal processing circuit 30 samples the current detection signal Vi (step S1004). Subsequently, the control circuit 36 included in the signal processing circuit 30 calculates the resistance value r1 of the thermistor Rd2 based on the digital value of the amplification signal Vamp3 (step S1005).

Then, the signal processing circuit 30 determines whether the resistance value r1 of the thermistor Rd2 is equal to or lower than a threshold value rt2 (step S1006). This determination corresponds to determining whether the target gas is switched from CO₂ gas to H₂ gas. That is, in a state where the thermistor Rd2 is maintained at the sixth temperature range, the resistance value r1 of the thermistor Rd2 decreases as the CO₂ gas concentration in the measurement atmosphere increases, or the resistance value r1 of the thermistor Rd2 remains almost unchanged with the CO₂ gas concentration in the measurement atmosphere, so that the resistance value r1 of the thermistor Rd2 should be equal to or smaller than the resistance value rt2 that is to be obtained when the CO₂ gas concentration is around the CO₂ gas concentration (e.g., 400 ppm) in the atmosphere under normal conditions. Therefore, when the condition that the resistance value r1 of the thermistor Rd2 is equal to or lower than the threshold value rt2 is satisfied (r1 ≤ rt2), the CO₂ gas concentration is calculated based on the gas detection signal Vgas (step S1007), and the output signal Vout corresponding to the CO₂ gas concentration is output to the outside (step S1008). Thereafter, the heating of the heaters MH1 and MH2 is stopped, and waiting is performed until the next measurement timing (step S1009). When the next measurement timing arrives, the flow returns to step S1001 for repetition of the above operation.

On the other hand, when the condition that the resistance value r1 of the thermistor Rd2 is equal to or smaller than the threshold value rt2 is not satisfied (r1 > rt2), it is suggested that H₂ gas is present at a high concentration in the measurement atmosphere. That is, when H₂ gas is present in the measurement atmosphere while the thermistor Rd2 is maintained at the sixth temperature range, the heat dissipation characteristics of the heater MH2 increase as the H₂ gas concentration increases, so that the temperature of the heater MH2 decreases and, consequently, the temperature of the thermistor Rd2 also decreases. Thus, the resistance value r1 of the thermistor Rd2 increases as the H₂ gas concentration in the measurement atmosphere increases. In this case, the H₂ gas concentration is calculated based on the gas detection signal Vgas (step S1010), and the output signal Vout corresponding to the H₂ gas concentration is output to the outside (step S1011). Thereafter, the heating of the heaters MH1 and MH2 is stopped, and waiting is performed until the next measurement timing (step S1009). When the next measurement timing arrives, the flow returns to step S1001 for repetition of the above operation.

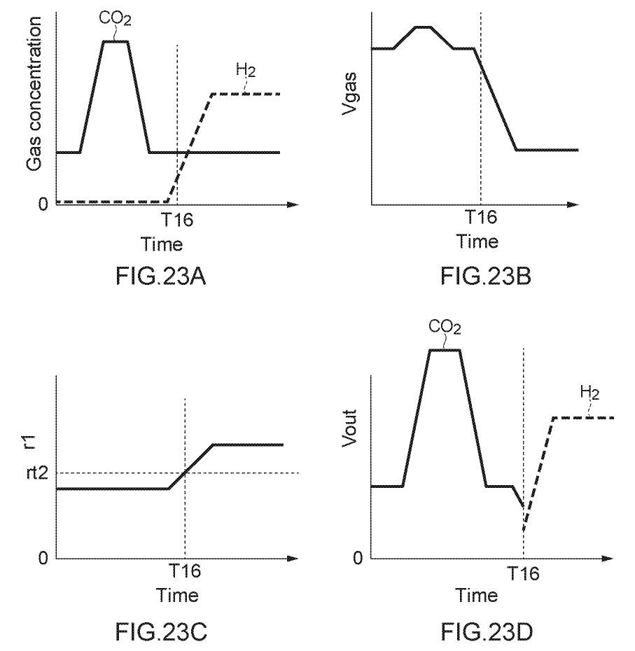

FIGS. 23A to 23D are graphs illustrating changes in the concentrations of H₂ gas and CO₂ gas, a change in the gas detection signal Vgas, a change in the resistance value r1 of the thermistor Rd2, and a change in the output signal Vout, respectively, in the second operation mode of the gas sensor 300.

In the example illustrated in FIG. 23A, after the CO₂ gas concentration temporarily changes, the H₂ gas concentration sharply increases around time T16. In this case, as illustrated in FIG. 23B, the level of the gas detection signal Vgas changes in association with the CO₂ gas concentration until time T16 and decreases at time T16. The decrease in the level of the gas detection signal Vgas is caused by the sharp increase in the H₂ gas concentration. Since the heat dissipation characteristics of the thermistor Rd2 increase as the H₂ gas concentration increases, the resistance value r1 of the thermistor Rd2 increases and exceeds the threshold value rt2 at time T16, as illustrated in FIG. 23C. As a result, as illustrated in FIG. 23D, the signal processing circuit 30 generates the output signal Vout indicating the CO₂ gas concentration until time T16 and generates the output signal Vout indicating the H₂ gas concentration after time T16.

As described above, in the second operation mode of the gas sensor 300, similarly to the second operation modes of the gas sensors 100 and 200, the CO₂ gas concentration in the measurement atmosphere is primarily measured, and when the resistance value r1 of the thermistor Rd2 exceeds the threshold value rt2 as a result of an increase in the H₂ gas concentration in the measurement atmosphere, the target gas is automatically switched from CO₂ gas to H₂ gas. Then, when the H₂ gas concentration in the measurement atmosphere decreases, the target gas is switched from H₂ gas to CO₂ gas. Further, in the second operation mode of the gas sensor 300, an increase in the H₂ gas concentration is detected by directly measuring the resistance value r1 of the thermistor Rd2, rather than the level of the gas detection signal Vgas, so that when the H₂ gas concentration increases, the target gas can be rapidly switched from CO₂ gas to H₂ gas. Note that, when the resistance value r1 of the thermistor Rd2 is measured, heating of the heater MH1 may be omitted.

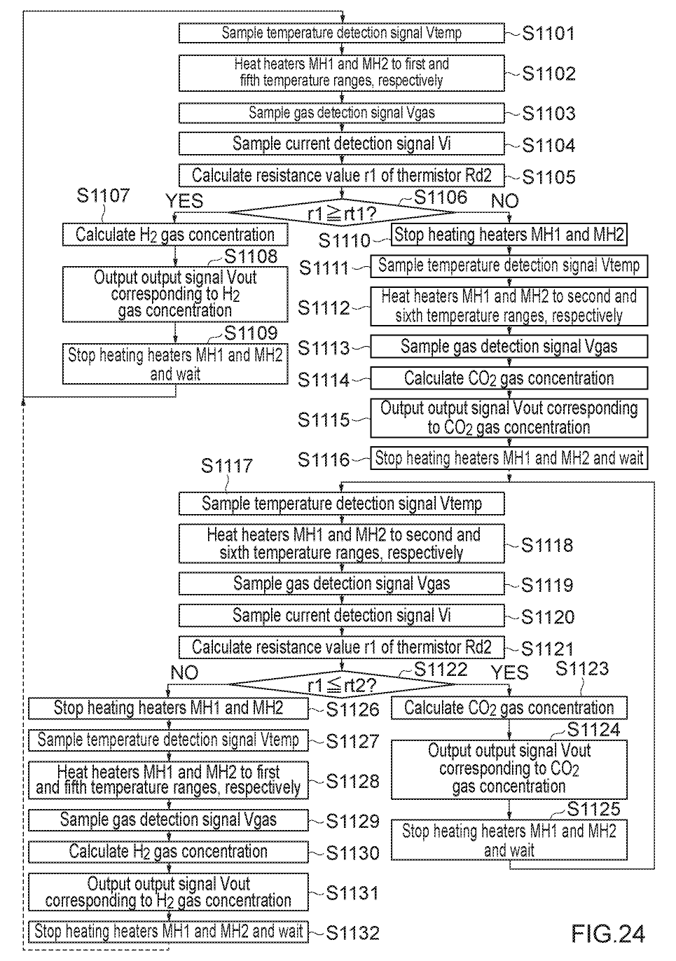

FIG. 24 is a flowchart illustrating a third operation mode of the gas sensor 300. The third operation mode of the gas sensor 300 is a mode in which the heaters MH1 and MH2 are heated to the first and fifth temperature ranges, respectively, when the H₂ gas concentration in the measurement atmosphere is measured, and to the second and sixth temperature ranges, respectively, when the CO₂gas concentration in the measurement atmosphere is measured.

First, in the third operation mode of the gas sensor 300, when the H₂ gas concentration in the measurement atmosphere is measured, the signal processing circuit 30 samples the temperature detection signal Vtemp to calculate the ambient temperature (step S1101). Subsequently, the control circuit 36 included in the signal processing circuit 30 outputs a heater instruction value calculated based on the ambient temperature to the DA converter 35 to heat the heaters MH1 and MH2 to the first and fifth temperature ranges, respectively (step S1102). The heater instruction value is converted into the heater voltages Vmh1 and Vmh2 by the DA converter 35, which are then applied to the heaters MH1 and MH2, respectively. Then, the signal processing circuit 30 samples the gas detection signal Vgas while the heaters MH1 and MH2 are maintained at the first and fifth temperature ranges, respectively (step S1103).

Then, the signal processing circuit 30 samples the current detection signal Vi (step S1104). Subsequently, the control circuit 36 included in the signal processing circuit 30 calculates the resistance value r1 of the thermistor Rd2 based on the digital value of the amplification signal Vamp3 (step S1105).

Then, the signal processing circuit 30 determines whether the resistance value r1 of the thermistor Rd2 is equal to or larger than the threshold value rt1 (step S1106). The operation performed when the condition that the resistance value r1 of the thermistor Rd2 is equal to or larger than the threshold value rt1 is satisfied (r1 ≥ rt1) is the same as that performed in the third operation mode of the gas sensor 200 described using FIG. 15. That is, the H₂ gas concentration is calculated based on the gas detection signal Vgas (step S1107), and the output signal Vout corresponding to the H₂ gas concentration is output to the outside (step S1108). Thereafter, the heating of the heaters MH1 and MH2 is stopped, and waiting is performed until the next measurement timing (step S1109). When the next measurement timing arrives, the flow returns to step S1101 for repetition of the above operation.

On the other hand, when the condition that resistance value r1 of the thermistor Rd2 is equal to or larger than the threshold value rt1 is not satisfied (r1 < rt1), the heating of the heaters MH1 and MH2 is temporarily stopped (step S1110), and the signal processing circuit 30 samples the temperature detection signal Vtemp again to calculate the ambient temperature (step S1111). Subsequently, the control circuit 36 included in the signal processing circuit 30 outputs a heater instruction value calculated based on the ambient temperature to the DA converter 35 to heat the heaters MH1 and MH2 to the second and sixth temperature ranges, respectively (step S1112). The heater instruction value is converted into the heater voltages Vmh1 and Vmh2 by the DA converter 35, which are then applied to the heaters MH1 and MH2, respectively. Then, the signal processing circuit 30 samples the gas detection signal Vgas while the heaters MH1 and MH2 are maintained at the second and sixth temperature ranges, respectively (step S1113).

Then, the CO₂ gas concentration is calculated based on the gas detection signal Vgas (step S1114), and the output signal Vout corresponding to the CO₂ gas concentration is output to the outside (step S1115). Thereafter, the heating of the heaters MH1 and MH2 is stopped, and waiting is performed until the next measurement timing (step S1116).

When the next measurement timing arrives, the signal processing circuit 30 samples the temperature detection signal Vtemp again to calculate the ambient temperature (step S1117). Subsequently, the control circuit 36 included in the signal processing circuit 30 outputs a heater instruction value calculated based on the ambient temperature to the DA converter 35 to heat the heaters MH1 and MH2 to the second and sixth temperature ranges, respectively (step S1118). The heater instruction value is converted into the heater voltages Vmh1 and Vmh2 by the DA converter 35, which are then applied to the heaters MH1 and MH2, respectively. Then, the signal processing circuit 30 samples the gas detection signal Vgas while the heaters MH1 and MH2 are maintained at the second and sixth temperature ranges, respectively (step S1119).

Then, the signal processing circuit 30 samples the current detection signal Vi (step S1120). Subsequently, the control circuit 36 included in the signal processing circuit 30 calculates the resistance value r1 of the thermistor Rd2 based on the digital value of the amplification signal Vamp3 (step S1121).

Then, the signal processing circuit 30 determines whether the resistance value r1 of the thermistor Rd2 is equal to or smaller than the threshold value rt2 (step S1122). The operation performed when the condition that the resistance value r1 of the thermistor Rd2 is equal to or smaller than the threshold value rt2 is satisfied (r1 ≤ rt2) is the same as that performed in the third operation mode of the gas sensor 200 described using FIG. 15. That is, the CO₂ gas concentration is calculated based on the gas detection signal Vgas (step S1123), and the output signal Vout corresponding to the CO₂ gas concentration is output to the outside (step S1124). Thereafter, the heating of the heaters MH1 and MH2 is stopped, and waiting is performed until the next measurement timing (step S1125). When the next measurement timing arrives, the flow returns to step S1117 for repetition of the above operation.

On the other hand, when the condition that the resistance value r1 of the thermistor Rd2 is equal to or smaller than the threshold value rt2 is not satisfied (r1 > rt2), the heating of the heaters MH1 and MH2 is temporarily stopped (step S1126), and the signal processing circuit 30 samples the temperature detection signal Vtemp again to calculate the ambient temperature (step S1127). Subsequently, the control circuit 36 included in the signal processing circuit 30 outputs a heater instruction value calculated based on the ambient temperature to the DA converter 35 to heat the heaters MH1 and MH2 to the first and fifth temperature ranges, respectively (step S1128). The heater instruction value is converted into the heater voltages Vmh1 and Vmh2 by the DA converter 35, which are then applied to the heaters MH1 and MH2, respectively. Then, the signal processing circuit 30 samples the gas detection signal Vgas while the heaters MH1 and MH2 are maintained at the first and fifth temperature ranges (step S1129).

Then, the H₂ gas concentration is calculated based on the gas detection signal Vgas (step S1130), and the output signal Vout corresponding to the H₂ gas concentration is output to the outside (step S1131). Thereafter, the heating of the heaters MH1 and MH2 is stopped, and waiting is performed until the next measurement timing (step S1132). When the next measurement timing arrives, the flow returns to step S1101 for repetition of the above operation.

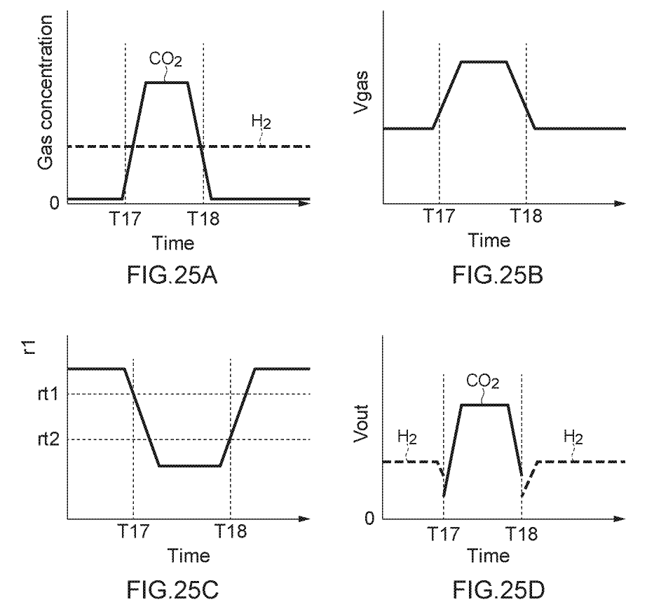

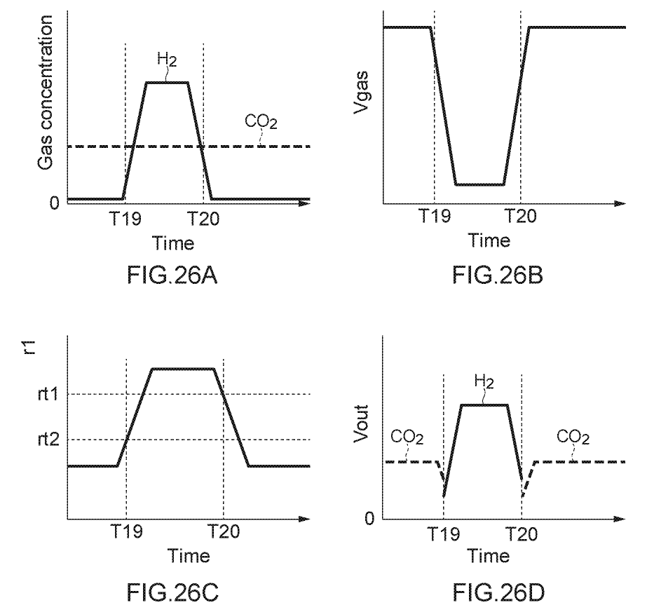

FIGS. 25A to 25D are graphs illustrating changes in the concentrations of H₂ gas and CO₂ gas, a change in the gas detection signal Vgas, a change in the resistance value r1 of the thermistor Rd2, and a change in the output signal Vout, respectively, in the third operation mode of the gas sensor 300. FIGS. 26A to 26D are graphs illustrating changes in the concentrations of H₂ gas and CO₂ gas, a change in the gas detection signal Vgas, a change in the resistance value r1 of the thermistor Rd2, and a change in the output signal Vout, respectively, in the third operation mode of the gas sensor 300.

In the example illustrated in FIG. 25A, the H₂ gas concentration is constant, whereas the CO₂ gas concentration sharply increases around time T17 and sharply decreases around time T18. In this case, as illustrated in FIG. 25B, the level of the gas detection signal Vgas increases between time T17 and time T18. The increase in the level of the gas detection signal Vgas is caused by the sharp increase in the CO₂ gas concentration. Since the heat dissipation characteristics of the thermistor Rd2 decrease as the CO₂ gas concentration increases, the resistance value r1 of the thermistor Rd2 decreases, falls below the threshold value rt1 at time T17, and then exceeds the threshold value rt2 at time T18, as illustrated in FIG. 25C. As a result, as illustrated in FIG. 25D, the signal processing circuit 30 generates the output signal Vout indicating the H₂gas concentration until time T17 by repeating steps S1101 to S1109, generates the output signal Vout indicating the CO₂ gas concentration from time T17 to T18 by repeating steps S1117 to S1125 after executing step S1110 to S1116, and generates again the output signal Vout indicting the H₂ gas concentration by repeating steps S1101 to S1109 after executing steps S1126 to S1132.

In the example illustrated in FIG. 26A, the CO₂ gas concentration is constant, whereas the H₂ gas concentration sharply increases around time T19 and sharply decreases around time T20. In this case, as illustrated in FIG. 26B, the level of the gas detection signal Vgas decreases between time T19 and time T20. The decrease in the level of the gas detection signal Vgas is caused by the sharp increase in the H₂ gas concentration. Since the heat dissipation characteristics of the thermistor Rd2 increase as the H₂ gas concentration increases, the resistance value r1 of the thermistor Rd2 increases, exceeds the threshold value rt2 at time T19, and then falls below the threshold value rt1 at time T20, as illustrated in FIG. 26C. As a result, as illustrated in FIG. 26D, the signal processing circuit 30 generates the output signal Vout indicating the CO₂ gas concentration until time T19 by repeating steps S1117 to S1125 after executing steps S1110 to S1116, generates the output signal Vout indicating the H₂ gas concentration from time T19 to time T20 by repeating steps S1101 to S1109 after executing steps S1126 to S1132, and generates again the output signal Vout indicting the CO₂ gas concentration by repeating steps S1117 to S1125 after executing steps S1110 to S1116.

As described above, in the third operation mode of the gas sensor 300, when the CO₂ gas concentration in the measurement atmosphere increases during the measurement of the H₂ gas concentration and the resistance value r1 of the thermistor Rd2 falls below the threshold value rt1, the target gas is automatically switched from H₂ gas to CO₂ gas; when the H₂ gas concentration in the measurement atmosphere increases during the measurement of the CO₂ gas concentration and the resistance value r1 of the thermistor Rd2 exceeds the threshold value rt2, the target gas is automatically switched from CO₂ gas to H₂ gas.

In the third operation mode of the gas sensor 300, during the measurement of H₂gas concentration, the heater MH1 is heated to the first temperature range optimized for the measurement of the H₂ gas concentration, and the heater MH2 is heated to the fifth temperature range in which a change in the heat dissipation characteristics in response to a change in the H₂gas concentration is small, allowing the H₂ gas concentration in the measurement atmosphere to be measured with high sensitivity and high accuracy as in the third operation mode of the gas sensor 200. Similarly, during the measurement of CO₂ gas, the heater MH1 is heated to the second temperature range optimized for the measurement of the CO₂ gas concentration, and the heater MH2 is heated to the sixth temperature range in which a change in the heat dissipation characteristics in response to a change in the CO₂ gas concentration is small, allowing the CO₂ gas concentration in the measurement atmosphere to be measured with high sensitivity and high accuracy as in the third operation mode of the gas sensor 200.

Further, in the third operation mode of the gas sensor 300, increases in the CO₂ gas concentration and the H₂ gas concentration are detected by directly measuring the resistance value r1 of the thermistor Rd2, rather than the level of the gas detection signal Vgas, so that when the H₂ gas concentration increases, the target gas can be rapidly switched from CO₂ gas to H₂ gas, and when the CO₂ gas concentration increases, the target gas can be rapidly switched from H₂ gas to CO₂ gas. Note that, when the resistance value r1 of the thermistor Rd2 is measured, heating of the heater MH1 may be omitted. Although, in the above example, the flow proceeds to step S1117 when the next measurement timing arrives in step S1116, the flow may proceed to step S1101 in this case.

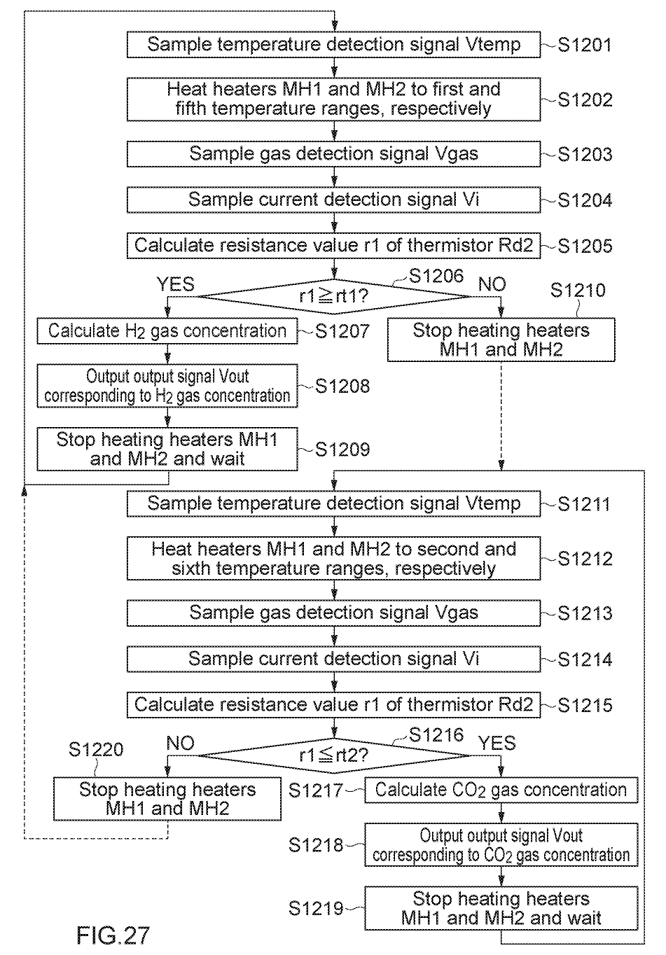

FIG. 27 is a flowchart illustrating a fourth operation mode of the gas sensor 300. In the fourth operation mode of the gas sensor 300, when the H₂ gas concentration in the measurement atmosphere is measured, the heaters MH1 and MH2 are heated to the first and fifth temperature ranges, respectively; whereas when the CO₂ gas concentration in the measurement atmosphere is measured, the heaters MH1 and MH2 are heated to the second and sixth temperature ranges, respectively, and a determination is made using the threshold value after the switching of the target gas and before the measurement of the concentration of the target gas.

First, in the fourth operation mode of the gas sensor 300, when the H₂ gas concentration in the measurement atmosphere is measured, the signal processing circuit 30 samples the temperature detection signal Vtemp to calculate the ambient temperature (step S1201). Subsequently, the control circuit 36 included in the signal processing circuit 30 outputs a heater instruction value calculated based on the ambient temperature to the DA converter 35 to heat the heaters MH1 and MH2 to the first and fifth temperature ranges, respectively (step S1202). The heater instruction value is converted into the heater voltages Vmh1 and Vmh2 by the DA converter 35, which are then applied to the heaters MH1 and MH2, respectively. Then, the signal processing circuit 30 samples the gas detection signal Vgas while the heaters MH1 and MH2 are maintained at the first and fifth temperature ranges, respectively (step S1203).

Then, the signal processing circuit 30 samples the current detection signal Vi (step S1204). Subsequently, the control circuit 36 included in the signal processing circuit 30 calculates the resistance value r1 of the thermistor Rd2 based on the digital value of the amplification signal Vamp3 (step S1205).

Then, the signal processing circuit 30 determines whether the resistance value r1 of the thermistor Rd2 is equal to or larger than the threshold value rt1 (step S1206). The operation performed when the condition that the resistance value r1 of the thermistor Rd2 is equal to or larger than the threshold value rt1 is satisfied (r1 ≥ rt1) is the same as that performed in the fourth operation mode of the gas sensor 200 described using FIG. 16. That is, the H₂ gas concentration is calculated based on the gas detection signal Vgas (step S1207), and the output signal Vout corresponding to the H₂ gas concentration is output to the outside (step S1208). Thereafter, the heating of the heaters MH1 and MH2 is stopped, and waiting is performed until the next measurement timing (step S1209). When the next measurement timing arrives, the flow returns to step S1201 for repetition of the above operation.

On the other hand, when the condition that the resistance value r1 of the thermistor Rd2 is equal to or larger than the threshold value rt1 is not satisfied (r1 < rt1), the heating of the heaters MH1 and MH2 is temporarily stopped (step S1210), and the target gas is switched from H₂ gas to CO₂ gas.

When the target gas is switched from H₂ gas to CO₂ gas, the signal processing circuit 30 samples the temperature detection signal Vtemp to calculate the ambient temperature (step S1211). Subsequently, the control circuit 36 included in the signal processing circuit 30 outputs a heater instruction value calculated based on the ambient temperature to the DA converter 35 to heat the heaters MH1 and MH2 to the second and sixth temperature ranges, respectively (step S1212). The heater instruction value is converted into the heater voltages Vmh1 and Vmh2 by the DA converter 35, which are applied to the heaters MH1 and MH2, respectively. Then, the signal processing circuit 30 samples the gas detection signal Vgas while the heaters MH1 and MH2 are maintained at the second and sixth temperature ranges, respectively (step S1213).

Then, the signal processing circuit 30 samples the current detection signal Vi (step S1214). Subsequently, the control circuit 36 included in the signal processing circuit 30 calculates the resistance value r1 of the thermistor Rd2 based on the digital value of the amplification signal Vamp3 (step S1215).

Then, the signal processing circuit 30 determines whether the resistance value r1 of the thermistor Rd2 is equal to or smaller than the threshold value rt2 (step S1216). The operation performed when the condition that the resistance value r1 of the thermistor Rd2 is equal to or smaller than the threshold value rt2 is satisfied (r1 ≤ rt2) is the same as that performed in the fourth operation mode of the gas sensor 200 described using FIG. 16. That is, the CO₂ gas concentration is calculated based on the gas detection signal Vgas (step S1217), and the output signal Vout corresponding to the CO₂ gas concentration is output to the outside (step S1218). Thereafter, the heating of the heaters MH1 and MH2 is stopped, and waiting is performed until the next measurement timing (step S1219). When the next measurement timing arrives, the flow returns to step S1211 for repetition of the above operation.

On the other hand, when the condition that the resistance value r1 of the thermistor Rd2 is equal to or smaller than the threshold value rt2 is not satisfied (r1 > rt2), the heating of the heaters MH1 and MH2 is temporarily stopped (step S1220), and the flow returns to step S1201, where the target gas is switched from CO₂ gas to H₂ gas.

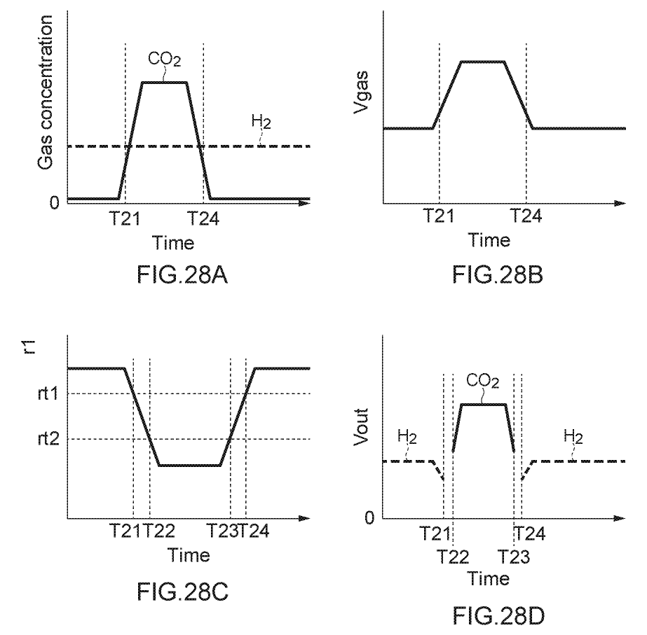

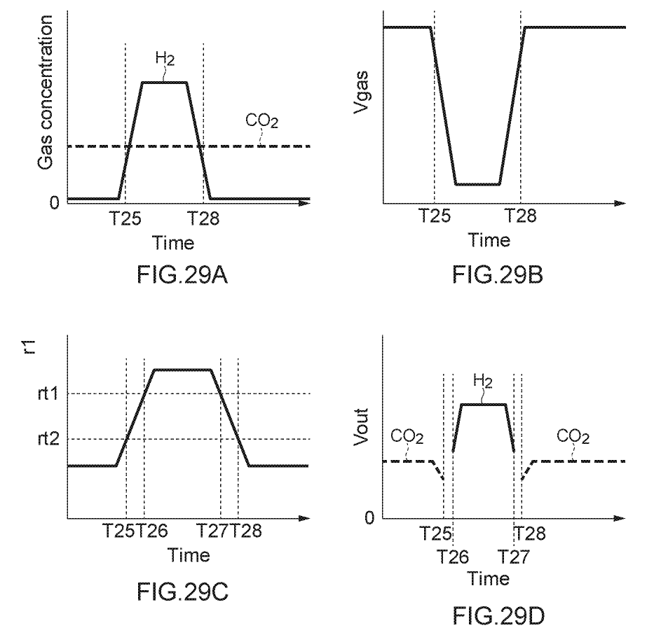

FIGS. 28A to 28D are graphs illustrating changes in the concentrations of H₂ gas and CO₂ gas, a change in the gas detection signal Vgas, a change in the resistance value r1 of the thermistor Rd2, and a change in the output signal Vout, respectively, in the third operation mode of the gas sensor 300. FIGS. 29A to 29D are graphs illustrating changes in the concentrations of H₂ gas and CO₂ gas, a change in the gas detection signal Vgas, a change in the resistance value r1 of the thermistor Rd2, and a change in the output signal Vout, respectively, in the third operation mode of the gas sensor 300.

In the example illustrated in FIG. 28A, the H₂ gas concentration is constant, whereas the CO₂ gas concentration sharply increases around time T21 and sharply decreases around time T24. In this case, as illustrated in FIG. 28B, the level of the gas detection signal Vgas increases between time T21 and time T24. The increase in the level of the gas detection signal Vgas is caused by the sharp increase in the CO₂ gas concentration. Since the heat dissipation characteristics of the thermistor Rd2 decrease as the CO₂ gas concentration increases, the resistance value r1 of the thermistor Rd2 decreases as illustrated in FIG. 28C. That is, the resistance value r1 of the thermistor Rd2 is equal to or larger than the threshold value rt1 until time T21; is smaller than the threshold value rt1 and larger than the threshold value rt2 from time T21 to time T22; is equal to or smaller than the threshold value rt2 from time T22 to time T23; is larger than the threshold value rt2 and smaller than the threshold value rt1 from time T23 to time 24; and is equal to or larger than the threshold value rt1 after time T24.

As a result, as illustrated in FIG. 28D, the signal processing circuit 30 generates the output signal Vout indicating the H₂gas concentration until time T21 by repeating steps S1201 to S1209, generates the output signal Vout indicating the CO₂ gas concentration from time T22 to time T23 by repeating steps S1211 to S1219, and generates again the output signal Vout indicating the H₂ gas concentration by repeating steps S1201 to S1209.

From time T21 to time T22, steps S1201 to S1206, steps S1210 to S1216, and step S1220 are repeated, during which no output signal Vout is obtained. Similarly, from time T23 to time T24, steps S1201 to S1206, steps S1210 to S1216, and step S1220 are repeated, during which no output signal Vout is obtained.

In the example illustrated in FIG. 29A, the CO₂ gas concentration is constant, whereas the H₂ gas concentration sharply increases around time T25 and sharply decreases around time T28. In this case, as illustrated in FIG. 29B, the level of the gas detection signal Vgas decreases between time T25 and time T28. The decrease in the level of the gas detection signal Vgas is caused by the sharp increase in the H₂ gas concentration. Since the heat dissipation characteristics of the thermistor Rd2 increase as the H₂ gas concentration increases, the resistance value r1 of the thermistor Rd2 increases as illustrated in FIG. 29C. That is, the resistance value r1 of the thermistor Rd2 is equal to or smaller than the threshold value rt2 until time T25; is smaller than the threshold value rt1 and larger than the threshold value rt2 from time T25 to time T26; is equal to or larger than the threshold value rt1 from time T26 to time T27; is larger than the threshold value rt2 and smaller than the threshold value rt1 from time T27 to time 28; and is equal to or larger than the threshold value rt2 after time T28.

As a result, as illustrated in FIG. 29D, the signal processing circuit 30 generates the output signal Vout indicating the CO₂gas concentration until time T25 by repeating steps S1211 to S1219, generates the output signal Vout indicating the H₂ gas concentration from time T26 to time T27 by repeating steps S1201 to S1209, and generates again the output signal Vout indicting the CO₂ gas concentration by repeating steps S1211 to S1219.

From time T25 to time T26, steps S1201 to S1206, steps S1210 to S1216, and step S1220 are repeated, during which no output signal Vout is obtained. Similarly, from time T27 to time T28, steps S1201 to S1206, steps S1210 to S1216, and step S1220 are repeated, during which no output signal Vout is obtained.

As described above, in the fourth operation mode of the gas sensor 300, the heater MH1 is heated to the first temperature range optimized for the measurement of the H₂ gas concentration during the measurement of H₂gas, and the heater MH2 is heated to the fifth temperature range in which a change in the heat dissipation characteristics in response to a change in the H₂gas concentration is small, allowing the H₂ gas concentration in the measurement atmosphere to be measured with high sensitivity and high accuracy. Similarly, the heater MH1 is heated to the second temperature range optimized for the measurement of the CO₂ gas concentration during the measurement of CO₂ gas, and the heater MH2 is heated to the sixth temperature range in which a change in the heat dissipation characteristics in response to a change in the CO₂ gas concentration is small, allowing the CO₂ gas concentration in the measurement atmosphere to be measured with high sensitivity and high accuracy.

In addition, in the fourth operation mode of the gas sensor 300, when the resistance value r1 of the thermistor Rd2 is smaller than the threshold value rt1 and larger than the threshold value rt2, both the calculation of the gas concentration and the generation of the output signal Vout are stopped. Thus, similarly to the fourth operation mode of the gas sensors 100 and 200, when the target gas is switched from H₂ gas to CO₂ gas, the output signal Vout indicating the CO₂ gas concentration can be output in a state where the CO₂ gas concentration is sufficiently high, and when the target gas is switched from CO₂ gas to H₂ gas, the output signal Vout indicating the H₂ gas concentration can be output in a state where the H₂ gas concentration is sufficiently high.

Further, in the fourth operation mode of the gas sensor 300, increases in the H₂ gas concentration and the CO₂ gas concentration are detected by directly measuring the resistance value r1 of the thermistor Rd2, rather than the level of the gas detection signal Vgas, so that when the H₂gas concentration increases, the target gas can be rapidly switched from CO₂ gas to H₂ gas, and when the CO₂ gas concentration increases, the target gas can be rapidly switched from H₂ gas to CO₂ gas. Note that, when the resistance value r1 of the thermistor Rd2 is measured, heating of the heater MH1 may be omitted.

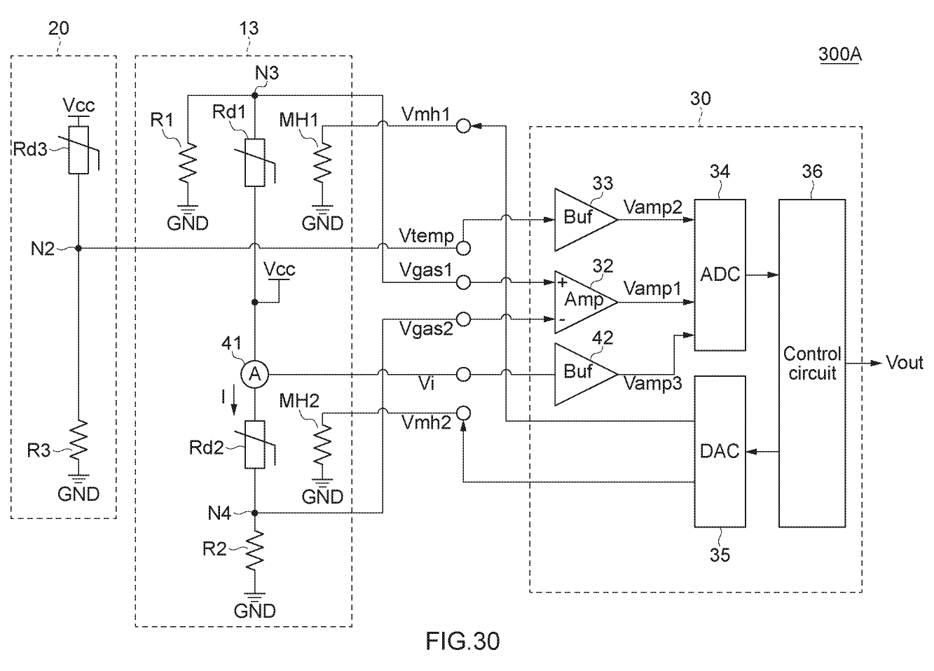

FIG. 30 is a circuit diagram illustrating the configuration of a gas sensor 300A according to a first modification of the third embodiment.

As illustrated in FIG. 30, the gas sensor 300A according to the first modification of the third embodiment differs from the gas sensor 200A according to the first modification of the second embodiment in that the current sensor 41 is connected in series with the thermistor Rd2 and that the buffer 42 is additionally provided in the signal processing circuit 30. Other basic configurations are the same as those of the gas sensor 200A according to the first modification of the second embodiment, so the same reference numerals are given to the same elements, and overlapping description will be omitted.

The current sensor 41 measures current flowing through the thermistor Rd2 to generate the current detection signal Vi. The buffer 42 buffers the current detection signal Vi to generate the amplification signal Vamp3. The amplification signal Vamp3 is converted into a corresponding digital value by the AD converter 34, which is then supplied to the control circuit 36. The control circuit 36 calculates the resistance value r1 of the thermistor Rd2 based on the digital value of the amplification signal Vamp3. Assuming that the value of a current flowing through the thermistor Rd2 is I, the resistance value r1 of the thermistor Rd2 can be calculated by the following equation: r1 = (Vcc – Vgas2)/I.

As exemplified by the gas sensor 300A according to the first modification of the third embodiment, also when the thermistors Rd1 and Rd2 are connected in parallel between the power supply Vcc and the ground GND, the target gas may be switched from one type to another based on the resistance value r1 of the thermistor Rd2 serving as a reference side element. The gas detection signal Vgas2, the current detection signal Vi, and the amplification signal Vamp3 are signals indicating the resistance value r1 of the thermistor Rd2 and derived from the thermistor Rd2.

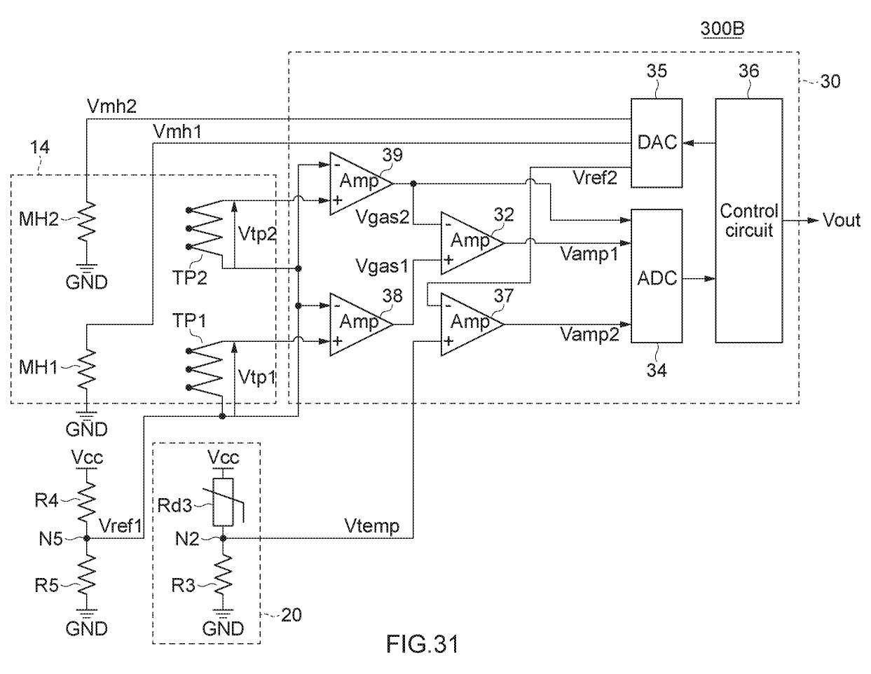

FIG. 31 is a circuit diagram illustrating the configuration of a gas sensor 300B according to a second modification of the third embodiment.

As illustrated in FIG. 31, the gas sensor 300B according to the second modification of the third embodiment differs from the gas sensor 200B according to the second modification of the second embodiment in that the gas detection signal Vgas2 output from the differential amplifier 39 is supplied to the AD converter 34. Other basic configurations are the same as those of the gas sensor 200B according to the second modification of the second embodiment, so the same reference numerals are given to the same elements, and overlapping description will be omitted.

The gas detection signal Vgas2 output from the differential amplifier 39 is converted into a corresponding digital value by the AD converter 34, which is then supplied to the control circuit 36. The control circuit 36 determines, based on the digital value of the gas detection signal Vgas2, which one of the concentration of H₂ gas and the concentration of CO₂ gas should be calculated.

That is, in the present embodiment, the gas detection signal Vgas2 is directly used for threshold determination. In the present embodiment, when the H₂ gas concentration increases, the level of the gas detection signal Vgas2 decreases; whereas when the CO₂ gas concentration increases, the level of the gas detection signal Vgas2 increases. Assuming that the level of the gas detection signal Vgas2 corresponding to the above-described threshold value rt1 is Vt1, and that the level of the gas detection signal Vgas2 corresponding to the above-described threshold value rt2 is Vt2, the above-described condition “r1 ≥ rt1” may be replaced with a condition “Vgas2 ≤ Vt1”, and the above-described condition “r1 ≤ rt2” may be replaced with a condition “Vgas2 ≥ Vt2”. That is, the H₂ gas concentration is calculated when Vgas2 ≤ Vt1, and the CO₂ gas concentration is calculated when Vgas2 ≥ Vt2.

As exemplified by the gas sensor 300B according to the second modification of the third embodiment, even when another type of temperature-sensing element, such as a thermopile element, is used, the target gas may be switched from one type to another based on the gas detection signal Vgas2, which is an output signal derived from the thermopile element TP2 serving as a reference side element.

While some embodiments of the technology according to the present disclosure have been described, the technology according to the present disclosure is not limited to the above embodiments, and various modifications may be made within the scope of the present disclosure, and all such modifications are included in the technology according to the present disclosure.

For example, in the gas sensor 100 according to the first embodiment, the gas concentration measurement is started from step S301 in the third operation mode and from step S401 in the fourth operation mode; however, the gas concentration measurement may be started from step S315 in the third operation mode and from step S409 in the fourth operation mode. In the gas sensor 200 according to the second embodiment, the gas concentration measurement is started from step S701 in the third operation mode and from step S801 in the fourth operation mode; however, the gas concentration measurement may be started from step S715 in the third operation mode and from step S809 in the fourth operation mode. In the gas sensor 300 according to the third embodiment, the gas concentration measurement is started from step S1101 in the third operation mode and from step S1201 in the fourth operation mode; however, the gas concentration measurement may be started from step S1117 in the third operation mode and from step S1211 in the fourth operation mode.

The technology according to the present disclosure includes the following configuration examples, but not limited thereto.

A gas sensor according to an aspect of the present disclosure includes: a first heater; a first temperature-sensing element whose temperature varies in response to a change in a temperature of the first heater ; and a signal processing circuit. The signal processing circuit is configured to: calculate a concentration of a first gas based on an output signal derived at least from the first temperature-sensing element obtained while the first heater is maintained at a first temperature range when a level of the output signal satisfies a first condition while the first heater is maintained at the first temperature range; and calculate a concentration of a second gas based on the output signal when the level of the output signal does not satisfy the first condition while the first heater is maintained at the first temperature range. The first gas and the second gas have opposite thermal conductivities with respect to air in the first temperature range. This allows the concentrations of a plurality of gases to be calculated.

In the above gas sensor, the signal processing circuit may be configured to calculate the concentration of the second gas based on the output signal obtained while the first heater is maintained at the first temperature range when the level of the output signal does not satisfy the first condition while the first heater is maintained at the first temperature range. This facilitates control by the signal processing circuit.

In the above gas sensor, the signal processing circuit may be configured to calculate the concentration of the second gas based on the output signal obtained while the first heater is maintained at a second temperature range different from the first temperature range when the level of the output signal does not satisfy the first condition while the first heater is maintained at the first temperature range. This allows the concentration of the second gas to be calculated more accurately.

In the above gas sensor, the first gas and the second gas may have opposite thermal conductivities with respect to air in the second temperature range, and the signal processing circuit may be configured to: heat the first heater to the second temperature range when the level of the output signal does not satisfy the first condition while the first heater is maintained at the first temperature range; and calculate the concentration of the second gas based on the output signal obtained while the first heater is maintained at the second temperature range when the level of the output signal satisfies a second condition while the first heater is maintained at the second temperature range. This allows the concentration of the second gas to be calculated in a state where the concentration of the second gas is sufficiently high.

In the above gas sensor, the first gas and the second gas may have opposite thermal conductivities with respect to air in the second temperature range, and the signal processing circuit may be configured to: calculate the concentration of the second gas based on the output signal obtained while the first heater is maintained at the second temperature range when the level of the output signal satisfies a second condition while the first heater is maintained at the second temperature range; and calculate the concentration of the first gas based on the output signal obtained while the first heater is maintained at the first temperature range when the level of the output signal does not satisfy the second condition while the first heater is maintained at the second temperature range. This allows the concentration of the first gas to be measured when the concentration of the first gas is high and allows the concentration of the second gas to be measured when the concentration of the second gas is high.

In the above gas sensor, the signal processing circuit may be configured to: heat the first heater to the first temperature range when the level of the output signal does not satisfy the second condition while the first heater is maintained at the second temperature range; and calculate the concentration of the first gas based on the output signal obtained while the first heater is maintained at the first temperature range when the level of the output signal satisfies the first condition while the first heater is maintained at the first temperature range. This allows the concentration of the first gas to be calculated in a state where the concentration of the first gas is sufficiently high.

The above gas sensor may further include a second heater and a second temperature-sensing element whose temperature varies in response to a change in the second heater, the output signal may be derived from the first and second temperature-sensing elements, and the signal processing circuit may be configured to: calculate the concentration of the first gas based on the output signal obtained while the first heater is maintained at the first temperature range and the second heater is maintained at a third temperature range different from the first and second temperature ranges when the level of the output signal satisfies the first condition while the first heater is maintained at the first temperature range and the second heater is maintained at the third temperature range; and calculate the concentration of the second gas based on the output signal when the level of the output signal does not satisfy the first condition while the first heater is maintained at the first temperature range and the second heater is maintained at the third temperature range. This allows the concentrations of the first and second gas to be measured more accurately.

In the above gas sensor, the signal processing circuit may be configured to calculate the concentration of the second gas based on the output signal obtained while the first heater is maintained at the first temperature range and the second heater is maintained at the third temperature range when the level of the output signal does not satisfy the first condition while the first heater is maintained at the first temperature range and the second heater is maintained at the third temperature range. This facilitates control by the signal processing circuit.

In the above gas sensor, the signal processing circuit may be configured to calculate the concentration of the second gas based on the output signal obtained while the first heater is maintained at the second temperature range and the second heater is maintained at a fourth temperature range different from the first and second temperature ranges when the level of the output signal does not satisfy the first condition while the first heater is maintained at the first temperature range and the second heater is maintained at the third temperature range. This allows the concentration of the second gas to be measured more accurately.

In the above gas sensor, the first gas and the second gas may have opposite thermal conductivities with respect to air in the second temperature range, and the signal processing circuit may be configured to: heat the first heater to the second temperature range and heat the second heater to the fourth temperature range when the level of the output signal does not satisfy the first condition while the first heater is maintained at the first temperature range and the second heater is maintained at the third temperature range; and calculate the concentration of the second gas based on the output signal obtained while the first heater is maintained at the second temperature range and the second heater is maintained at the fourth temperature range when the level of the output signal satisfies a second condition while the first heater is maintained at the second temperature range and the second heater is maintained at the fourth temperature range. This allows the concentration of the second gas to be calculated in a state where the concentration of the second gas is sufficiently high.

In the above gas sensor, the first gas and the second gas may have opposite thermal conductivities with respect to air in the second temperature range, and the signal processing circuit may be configured to: calculate the concentration of the second gas based on the output signal obtained while the first heater is maintained at the second temperature range and the second heater is maintained at the fourth temperature range when the level of the output signal satisfies the second condition while the first heater is maintained at the second temperature range and the second heater is maintained at the fourth temperature range; and calculate the concentration of the first gas based on the output signal obtained while the first heater is maintained at the first temperature range and the second heater is maintained at the third temperature range when the level of the output signal does not satisfy the second condition while the first heater is maintained at the second temperature range and the second heater is maintained at the fourth temperature range. This allows the concentration of the first gas to be measured when the concentration of the first gas is high and allows the concentration of the second gas to be measured when the concentration of the second gas is high.

In the above gas sensor, the signal processing circuit may be configured to: heat the first heater to the first temperature range and heat the second heater to the third temperature range when the level of the output signal does not satisfy the second condition while the first heater is maintained at the second temperature range and the second heater is maintained at the fourth temperature range; and calculate the concentration of the first gas based on the output signal obtained while the first heater is maintained at the first temperature range and the second heater is maintained at the third temperature range when the level of the output signal satisfies the first condition while the first heater is maintained at the first temperature range and the second heater is maintained at the third temperature range. This allows the concentration of the first gas to be calculated in a state where the concentration of the first gas is sufficiently high.

A gas sensor according to another aspect of the present disclosure includes: a first heater; a second heater; a first temperature-sensing element whose temperature varies in response to a change in a temperature of the first heater; a second temperature-sensing element whose temperature varies in response to a change in a temperature of the second heater; and a signal processing circuit. The signal processing circuit may be configured to: calculate a concentration of a first gas based on a first output signal derived from the first temperature-sensing element and the second temperature-sensing element while the first heater is maintained at a first temperature range and the second heater is maintained at a fifth temperature range different from the first temperature range when a level of a second output signal derived from the second temperature-sensing element satisfies a first condition while the second heater is maintained at the fifth temperature range; and calculate a concentration of a second gas based on the first output signal when a level of the second output signal does not satisfy the first condition while the second heater is maintained at the fifth temperature range. A thermal conductivity of the first gas in the fifth temperature range and a thermal conductivity of the second gas in the fifth temperature range are different from each other. This allows the concentrations of a plurality of gases to be calculated.

In the above gas sensor, the signal processing circuit may be configured to calculate the concentration of the second gas based on the first output signal obtained while the first heater is maintained at the first temperature range and the the second heater is maintained at the fifth temperature range when the level of the second output signal does not satisfy the first condition while the second heater is maintained at the fifth temperature range. This facilitates control by the signal processing circuit.

In the above gas sensor, the signal processing circuit may be configured to calculate the concentration of the second gas based on the first output signal obtained while the first heater is maintained at a second temperature range different from the first and fifth temperature ranges and the the second heater is maintained at a sixth temperature range different from the first, second, and fifth temperature ranges when the level of the second output signal does not satisfy the first condition while the second heater is maintained at the fifth temperature range. This allows the concentration of the second gas to be calculated more accurately.

In the above gas sensor, a thermal conductivity of the first gas in the sixth temperature range and a thermal conductivity of the second gas in the sixth temperature range may be different from each other, and the signal processing circuit may be configured to: heat the second heater to the sixth temperature range when the level of the second output signal does not satisfy the first condition while the second heater is maintained at the fifth temperature range; and calculate the concentration of the second gas based on the first output signal obtained while the first heater is maintained at the second temperature range and the second heater is maintained at the sixth temperature range when the level of the second output signal satisfies a second condition while the second heater is maintained at the sixth temperature range. This allows the concentration of the second gas to be calculated in a state where the concentration of the second gas is sufficiently high.

In the above gas sensor, a thermal conductivity of the first gas in the sixth temperature range and a thermal conductivity of the second gas in the sixth temperature range may be different from each other, and the signal processing circuit may be configured to: calculate the concentration of the second gas based on the first output signal obtained when the first heater is maintained at the second temperature range and the second heater is maintained at the sixth temperature range when the level of the second output signal satisfies a second condition while the second heater is maintained at the sixth temperature range; and calculate the concentration of the first gas based on the first output signal obtained while the first heater is maintained at the first temperature range and the second heater is maintained at the fifth temperature range when the level of the second output signal does not satisfy the second condition while the second heater is maintained at the sixth temperature range. This allows the concentration of the first gas to be measured when the concentration of the first gas is high and allows the concentration of the second gas to be measured when the concentration of the second gas is high.

In the above gas sensor, the the signal processing circuit may be configured to: eat the second heater to the fifth temperature range when the level of the second output signal does not satisfy the second condition while the second heater is maintained at the sixth temperature range; and calculate the concentration of the first gas based on the first output signal obtained while the first heater is maintained at the first temperature range and the second heater is maintained at the fifth temperature range when the level of the second output signal satisfies the first condition while the second heater is maintained at the fifth temperature range. This allows the concentration of the first gas to be calculated in a state where the concentration of the first gas is sufficiently high.

What is claimed is:

1. A gas sensor comprising:
a first heater;
a first temperature-sensing element whose temperature varies in response to a change in a temperature of the first heater; and
a signal processing circuit,
wherein the signal processing circuit is configured to:
calculate a concentration of a first gas based on an output signal derived at least from the first temperature-sensing element obtained while the first heater is maintained at a first temperature range when a level of the output signal satisfies a first condition while the first heater is maintained at the first temperature range; and
calculate a concentration of a second gas based on the output signal when the level of the output signal does not satisfy the first condition while the first heater is maintained at the first temperature range, and
wherein the first gas and the second gas have opposite thermal conductivities with respect to air in the first temperature range.

2. The gas sensor as claimed in claim 1, wherein the signal processing circuit is configured to calculate the concentration of the second gas based on the output signal obtained while the first heater is maintained at the first temperature range when the level of the output signal does not satisfy the first condition while the first heater is maintained at the first temperature range.

3. The gas sensor as claimed in claim 1, wherein the signal processing circuit is configured to calculate the concentration of the second gas based on the output signal obtained while the first heater is maintained at a second temperature range different from the first temperature range when the level of the output signal does not satisfy the first condition while the first heater is maintained at the first temperature range.

4. The gas sensor as claimed in claim 3,
wherein the first gas and the second gas have opposite thermal conductivities with respect to air in the second temperature range,
wherein the signal processing circuit is configured to:
heat the first heater to the second temperature range when the level of the output signal does not satisfy the first condition while the first heater is maintained at the first temperature range; and
calculate the concentration of the second gas based on the output signal obtained while the first heater is maintained at the second temperature range when the level of the output signal satisfies a second condition while the first heater is maintained at the second temperature range.

5. The gas sensor as claimed in claim 3,
wherein the first gas and the second gas have opposite thermal conductivities with respect to air in the second temperature range, and
wherein the signal processing circuit is configured to:
calculate the concentration of the second gas based on the output signal obtained while the first heater is maintained at the second temperature range when the level of the output signal satisfies a second condition while the first heater is maintained at the second temperature range; and
calculate the concentration of the first gas based on the output signal obtained while the first heater is maintained at the first temperature range when the level of the output signal does not satisfy the second condition while the first heater is maintained at the second temperature range.

6. The gas sensor as claimed in claim 4, wherein the signal processing circuit is configured to:
heat the first heater to the first temperature range when the level of the output signal does not satisfy the second condition while the first heater is maintained at the second temperature range; and
calculate the concentration of the first gas based on the output signal obtained while the first heater is maintained at the first temperature range when the level of the output signal satisfies the first condition while the first heater is maintained at the first temperature range.

7. The gas sensor as claimed in claim 1, further comprising:
a second heater; and
a second temperature-sensing element whose temperature varies in response to a change in the second heater,
wherein the output signal is derived from the first temperature-sensing element and the second temperature-sensing element, and
wherein the signal processing circuit is configured to:
calculate the concentration of the first gas based on the output signal obtained while the first heater is maintained at the first temperature range and the second heater is maintained at a third temperature range different from the first and second temperature ranges when the level of the output signal satisfies the first condition while the first heater is maintained at the first temperature range and the second heater is maintained at the third temperature range; and
calculate the concentration of the second gas based on the output signal when the level of the output signal does not satisfy the first condition while the first heater is maintained at the first temperature range and the second heater is maintained at the third temperature range.

8. The gas sensor as claimed in claim 7, wherein the signal processing circuit is configured to calculate the concentration of the second gas based on the output signal obtained while the first heater is maintained at the first temperature range and the second heater is maintained at the third temperature range when the level of the output signal does not satisfy the first condition while the first heater is maintained at the first temperature range and the second heater is maintained at the third temperature range.

9. The gas sensor as claimed in claim 7, wherein the signal processing circuit is configured to calculate the concentration of the second gas based on the output signal obtained while the first heater is maintained at the second temperature range and the second heater is maintained at a fourth temperature range different from the first and second temperature ranges when the level of the output signal does not satisfy the first condition while the first heater is maintained at the first temperature range and the second heater is maintained at the third temperature range.

10. The gas sensor as claimed in claim 9,
wherein the first gas and the second gas have opposite thermal conductivities with respect to air in the second temperature range, and
wherein the signal processing circuit is configured to:
heat the first heater to the second temperature range and heat the second heater to the fourth temperature range when the level of the output signal does not satisfy the first condition while the first heater is maintained at the first temperature range and the second heater is maintained at the third temperature range; and
calculate the concentration of the second gas based on the output signal obtained while the first heater is maintained at the second temperature range and the second heater is maintained at the fourth temperature range when the level of the output signal satisfies a second condition while the first heater is maintained at the second temperature range and the second heater is maintained at the fourth temperature range.

11. The gas sensor as claimed in claim 9,
wherein the first gas and the second gas have opposite thermal conductivities with respect to air in the second temperature range, and
wherein the signal processing circuit is configured to:
calculate the concentration of the second gas based on the output signal obtained while the first heater is maintained at the second temperature range and the second heater is maintained at the fourth temperature range when the level of the output signal satisfies the second condition while the first heater is maintained at the second temperature range and the second heater is maintained at the fourth temperature range; and
calculate the concentration of the first gas based on the output signal obtained while the first heater is maintained at the first temperature range and the second heater is maintained at the third temperature range when the level of the output signal does not satisfy the second condition while the first heater is maintained at the second temperature range and the second heater is maintained at the fourth temperature range.

12. The gas sensor as claimed in claim 10, wherein the signal processing circuit is configured to:
heat the first heater to the first temperature range and heat the second heater to the third temperature range when the level of the output signal does not satisfy the second condition while the first heater is maintained at the second temperature range and the second heater is maintained at the fourth temperature range; and
calculate the concentration of the first gas based on the output signal obtained while the first heater is maintained at the first temperature range and the second heater is maintained at the third temperature range when the level of the output signal satisfies the first condition while the first heater is maintained at the first temperature range and the second heater is maintained at the third temperature range.

13. A gas sensor comprising:
a first heater;
a second heater;
a first temperature-sensing element whose temperature varies in response to a change in a temperature of the first heater;
a second temperature-sensing element whose temperature varies in response to a change in a temperature of the second heater; and
a signal processing circuit,
wherein the signal processing circuit is configured to:
calculate a concentration of a first gas based on a first output signal derived from the first temperature-sensing element and the second temperature-sensing element while the first heater is maintained at a first temperature range and the second heater is maintained at a fifth temperature range different from the first temperature range when a level of a second output signal derived from the second temperature-sensing element satisfies a first condition while the second heater is maintained at the fifth temperature range; and
calculate a concentration of a second gas based on the first output signal when a level of the second output signal does not satisfy the first condition while the second heater is maintained at the fifth temperature range, and
wherein a thermal conductivity of the first gas in the fifth temperature range and a thermal conductivity of the second gas in the fifth temperature range are different from each other.

14. The gas sensor as claimed in claim 13, wherein the signal processing circuit is configured to calculate the concentration of the second gas based on the first output signal obtained while the first heater is maintained at the first temperature range and the the second heater is maintained at the fifth temperature range when the level of the second output signal does not satisfy the first condition while the second heater is maintained at the fifth temperature range.

15. The gas sensor as claimed in claim 13, wherein the signal processing circuit is configured to calculate the concentration of the second gas based on the first output signal obtained while the first heater is maintained at a second temperature range different from the first and fifth temperature ranges and the the second heater is maintained at a sixth temperature range different from the first, second, and fifth temperature ranges when the level of the second output signal does not satisfy the first condition while the second heater is maintained at the fifth temperature range.

16. The gas sensor as claimed in claim 15,
wherein a thermal conductivity of the first gas in the sixth temperature range and a thermal conductivity of the second gas in the sixth temperature range are different from each other, and
wherein the signal processing circuit is configured to:
heat the second heater to the sixth temperature range when the level of the second output signal does not satisfy the first condition while the second heater is maintained at the fifth temperature range; and
calculate the concentration of the second gas based on the first output signal obtained while the first heater is maintained at the second temperature range and the second heater is maintained at the sixth temperature range when the level of the second output signal satisfies a second condition while the second heater is maintained at the sixth temperature range.

17. The gas sensor as claimed in claim 15,
wherein a thermal conductivity of the first gas in the sixth temperature range and a thermal conductivity of the second gas in the sixth temperature range are different from each other, and
wherein the signal processing circuit is configured to:
calculate the concentration of the second gas based on the first output signal obtained when the first heater is maintained at the second temperature range and the second heater is maintained at the sixth temperature range when the level of the second output signal satisfies a second condition while the second heater is maintained at the sixth temperature range; and
calculate the concentration of the first gas based on the first output signal obtained while the first heater is maintained at the first temperature range and the second heater is maintained at the fifth temperature range when the level of the second output signal does not satisfy the second condition while the second heater is maintained at the sixth temperature range.

18. The gas sensor as claimed in claim 16, wherein the the signal processing circuit is configured to:
heat the second heater to the fifth temperature range when the level of the second output signal does not satisfy the second condition while the second heater is maintained at the sixth temperature range; and
calculate the concentration of the first gas based on the first output signal obtained while the first heater is maintained at the first temperature range and the second heater is maintained at the fifth temperature range when the level of the second output signal satisfies the first condition while the second heater is maintained at the fifth temperature range.
